(19) United States
(12) Patent Application Publication
(10) Pub. No.: US 2022/0036974 A1
(43) Pub. Date: Feb. 3, 2022

(54) Pooled testing methods using compressed sensing for increasing the throughput and reliability of tests for the detection of defective units in a population

(71) Applicants: University of Iowa Research Foundation, Iowa City, IA (US); The Penn State Research Foundation, University Park, PA (US)

(72) Inventors: Weiyu Xu, Iowa City, IA (US); Xiaodong Wu, Iowa City, IA (US); Jirong Yi, Iowa City, IA (US); Raghu Mudumbai, Iowa City, IA (US); Myung Cho, Erie, PA (US)

(73) Assignees: University of Iowa Research Foundation, Iowa City, IA (US); The Penn State Research Foundation, University Park, PA (US)

(21) Appl. No.: 17/387,863

(22) Filed: Jul. 28, 2021

Related U.S. Application Data
(60) Provisional application No. 63/057,721, filed on Jul. 28, 2020.

(51) Int. Cl. G16B 40/00; G16H 10/40; C12Q 1/70; G01N 33/68

(52) U.S. Cl. CPC G16B 40/00; G01N 33/6854; C12Q 1/70; G16H 10/40

(57) ABSTRACT

A method for pooled sample testing for a target substance using compressed sensing includes receiving a plurality of individual samples, determining a mixing matrix for a plurality of pooled sample mixtures to create by mixing portions of selected ones of the plurality of individual samples, and determining an allocation matrix for the plurality of pooled samples, wherein the allocation matrix allocations portions of each of the plurality of pooled samples for each test, performing mixing to create the plurality of pooled sample mixtures based on the mixing matrix and the allocation matrix, performing quantitative tests on the plurality of pooled sample mixtures so as to estimate an amount of the target substance contained within each of the plurality of pooled sample mixtures, and decoding results of the quantitative tests to determine quantitative estimates of amount of the target substance in each of the plurality of individual samples.

### RELATED APPLICATION

This application claims priority to U.S. Provisional Patent Application No. 63/057,721, filed Jul. 28, 2020, hereby incorporated by reference in its entirety.

### GRANT REFERENCE

This invention was made with government support under NSF 2031218 awarded by the National Science Foundation. The government has certain rights in the invention.

### FIELD OF THE INVENTION

The present invention relates to pooled testing methods. More particularly, but not exclusively, the present invention relates to methods and systems for diagnostic testing to identify a small number of defective units in a large population using as few tests as possible. Furthermore, this method is capable of providing accurate diagnostics for each individual in the population even if the tests used are inaccurate.

### BACKGROUND

A simple version called “group testing” of this idea has been around since World War II and is now well-accepted in infectious disease diagnostics work like this: instead of testing individual samples one by one, a pooled mixture of samples from many individuals is tested together for the presence of a defect or pathogen. If the result is negative, we can immediately infer that all individuals in that pool are defect-free. Only when the result of the pooled test is positive, do we need to test individual samples.

When the fraction of defective units in the population is small, this can lead to a significant reduction in the number of tests required. However, this method has some drawbacks. First, the mixing process can damage or contaminate test samples which can cause false positives and/or negatives thereby reducing the accuracy and reliability of the test results. Secondly, any test error can be costly: a single test error may result in an inaccurate diagnosis for many individuals.

What is needed are methods and systems that use mathematically sophisticated sample mixing and post-processing that substantially improve on group testing both in terms of further reducing the number of tests required and increasing the diagnostic accuracy even if the individual tests are error prone.

### SUMMARY

Therefore, it is a primary object, feature, or advantage of the present invention to improve over the state of the art.

It is a further object, feature, or advantage of the present invention to use compressed sensing to increase the throughput and reliability of diagnostic tests.

It is a still further object, feature, or advantage to provide for group testing which uses non-binary diagnostics tests.

Another object, feature, or advantage is to produce quantitative estimates of the amount of a target substance found in a pooled test sample.

Another object, feature, or advantage is to produce quantitative estimates of the amount of a target substance found in individual samples.

Another object, feature, or advantage is to provide error correcting capability to increase the diagnostic accuracy of test results without performing more tests.

Yet another object, feature, or advantage is to provide adaptive error correction.

Another object, feature, or advantage is to provide a certificate of accuracy of the final test results.

Yet another object, feature, or advantage is to provide novel computational algorithms for decoding pooled sample test results.

One or more of these and/or other objects, features, or advantages of the present invention will become apparent from the specification and claims that follow. No single embodiment need provide each and every object, feature, or advantage. Different embodiments may have different objects, features, or advantages. Therefore, the present invention is not to be limited to or by any objects, features, or advantages stated herein.

According to one aspect, a method for pooled sample testing for a target substance using compressed sensing is provided. The method includes receiving a plurality of individual samples, determining a mixing matrix for a plurality of pooled sample mixtures to create by mixing portions of selected ones of the plurality of individual samples, and determining an allocation matrix for the plurality of pooled samples, wherein the allocation matrix allocations portions of each of the plurality of pooled samples for each test. The method further includes performing mixing to create the plurality of pooled sample mixtures based on the mixing matrix and the allocation matrix. The method further includes performing quantitative tests on the plurality of pooled sample mixtures so as to estimate an amount of the target substance contained within each of the plurality of pooled sample mixtures. The method further includes decoding results of the quantitative tests on the plurality of the pooled sample mixtures using the mixing matrix and the allocation matrix to determine quantitative estimates of amount of the target substance in each of the plurality of individual samples.

According to another aspect, a system pooled sample testing for a target substance using compressed sensing includes a computing device having a memory, instructions stored on the memory for: determining a mixing matrix for a plurality of pooled sample mixtures to create by mixing portions of selected ones of a plurality of individual samples; determining an allocation matrix for the plurality of pooled samples, wherein the allocation matrix allocations portions of each of the plurality of pooled samples for each test; and decoding results of the quantitative tests on the plurality of the pooled sample mixtures using the mixing matrix and the allocation matrix to determine quantitative estimates of amount of the target substance in each of the plurality of individual samples.

According to another aspect, a method for pooled sample testing for a target substance using adaptive compressed sensing is provided. The method includes allocating portions of a plurality of individual samples and mixing the portions to provide pooled sample tests, performing quantitative testing on the pooled sample tests to provide test results, and analyzing the test results and performing additional allocation of portions of the plurality of individual samples and mixing of the portions to provide at least one additional pooled sample test.

### BRIEF DESCRIPTION OF THE DRAWINGS

Illustrated embodiments of the disclosure are described in detail below with reference to the attached drawing figures, which are incorporated by reference herein.

FIG. 1 is a pictorial representation providing an overview of a pooled testing method using compressed sensing.

FIG. 2 is a pictorial representation of a system.

FIG. 3 provides amplification plots of real-time polymerase chain reaction (PCR) taken from [37]. According to [37], this figure is about “Relative fluorescence vs. cycle number.” “Amplification plots are created when the fluorescent signal from each sample is plotted against cycle number; therefore, amplification plots represent the accumulation of product over the duration of the real-time PCR experiment. The samples used to create the plots in this figure are a dilution series of the target DNA sequence.” [37]

FIG. 4: n=60; k=3. Binary measurement matrix with entries i.i.d. according to Bernoulli distribution.

FIG. 5: n=60; k=5. Binary measurement matrix with entries i.i.d. according to Bernoulli distribution.

FIG. 6: n=120; k=3. Binary measurement matrix with entries i.i.d. according to Bernoulli distribution.

FIG. 7: n=120; k=5. Binary measurement matrix with entries i.i.d. according to Bernoulli distribution.

FIG. 8: n=60; k=3. Expander measurement matrix with 5 ‘1’ s in each column.

FIG. 9: n=60; k=5. Expander measurement matrix with 5 ‘1’s in each column.

FIG. 10: n=120; k=3. Expander measurement matrix with 5 ‘1’ sin each column.

FIG. 11: n=120; k=5. Expander measurement matrix with 5 ‘1’s in each column.

FIG. 12: n=200; k=2. Expander measurement matrix with 5 ‘1’s in each column. Noisy measurements.

FIG. 13 provides exhaustive search for binary measurement matrix with entries from Bernoulli distribution. The magnitude of the noise vector is set at 10⁻³.

FIG. 14 provides the Rates versus Number of People Tested n. The number of pooling measurement is m=6, and k≈0.087×n persons carry viruses. Binary measurement matrix with entries i.i.d. according to Bernoulli distribution.

FIG. 15: n=60; k=3. Binary measurement matrix with entries i.i.d. according to Bernoulli distribution. Noisy measurements.

FIG. 16: n=60; k=5. Binary measurement matrix with entries i.i.d. according to Bernoulli distribution. Noisy measurements.

FIG. 17: n=120; k=3. Binary measurement matrix with entries i.i.d. according to Bernoulli distribution. Noisy measurements.

FIG. 18: n=120; k=5. Binary measurement matrix with entries i.i.d. according to Bernoulli distribution. Noisy measurements.

FIG. 19: n=60; k=3. Expander measurement matrix with 5 ‘1’s in each column. Noisy measurements.

FIG. 20: n=60; k=5. Expander measurement matrix with 5 ‘1’s in each column. Noisy measurements.

FIG. 21: n=120; k=3. Expander measurement matrix with 5 ‘1’s in each column. Noisy measurements.

FIG. 22: n=120; k=5. Expander measurement matrix with 5 ‘1’s in each column. Noisy measurements.

FIG. 23 provides the Overall procedure of Covid-19 testing using IDT primers and probes [24].

FIG. 24 provides a conceptual illustration of efficient group testing via compressed sensing.

FIG. 25A to FIG. 25F provide the False Negative Rate (FNR) and the corresponding False Positive Rate (FPR) with n=25, k=3, and Gaussian noise level 1 e0. FIG. 25A: FNR (Pout=0.01). FIG. 25B: FNR (Pout=0.05). FIG. 25C: FNR (Pout=0.15). FIG. 25D: FPR (Pₒᵤₜ=0.01). FIG. 25E: FPR (Pₒᵤₜ=0.05). FIG. 25F: FPR (Pₒᵤₜ=0.15).

FIG. 26A to FIG. 26F provide the False Negative Rate (FNR) and the corresponding False Positive Rate (FPR) with n=40, k=3, and Gaussian noise level 1e0. FIG. 26A: FNR (Pout=0.01). FIG. 26B: FNR (Pout=0.05). FIG. 26C: FNR (Pout=0.15). FIG. 26D: FPR (Pout=0.01). FIG. 26E: FPR (Pout t=0.05). FIG. 26F: FPR (Pout=0.15).

FIG. 27A to FIG. 27F provide the False Negative Rate (FNR) and the corresponding False Positive Rate (FPR) with n=25, k=3, Pout=0.05, and noise level varied from 5e-1 to 2e0. FIG. 27A: FNR (Noise level: Se-1). FIG. 27B: FNR (Noise level: 1e0). FIG. 27C: FNR (Noise level: 2e0). FIG. 27D: FPR (Noise level: Se-I). FIG. 27E: FPR (Noise level: 1e0). FIG. 27F: FPR (Noise level: 2e0).

FIG. 28A, FIG. 28B, FIG. 28C, FIG. 28D provide optimized group testing mixing matrix design. FIG. 29A, FIG. 29B, FIG. 29C provide Hamming code parity check pooling matrix design for N=7 (FIG. 28A), 15 (FIG. 28B), and 31 (FIG. 28C). FIG. 28A: N=7 numerical matrix with 3 pools (3×7). FIG. 28B: N=15 numerical matrix with 4 pools (4×15). FIG. 28C: N=31 pixel matrix with 5 pools (5×31). FIG. 28D: Bipartite pooling matrix design optimized for high N and prevalence rates. N=40 pixel matrix with 16 pools (16×40). FIG. 28A and FIG. 28B disclose 1 indicates patient is included in the pool. 0 indicates the patient is not included in the pool. FIG. 28C and FIG. 28D disclose white pixel indicates patient included in pool. Black pixel indicates patient not included in pool.

FIG. 29A and FIG. 29B provide modified pooling protocol eliminates dilution effect of group testing. FIG. 29A: RNA extraction and qRT-PCR workflow in individual testing, traditional pooling (group testing), and the modified pooling protocol. Numerical examples are theoretical to display dilution effect and can be scaled to individual diagnostic testing facility protocols. FIG. 29B: MHV-1 was used to generate individual samples of various viral loads (1×109-1×102 copy number/qRT-PCR reaction). qRT-PCR was performed on each samples to develop ground truth Ct values. Samples were then used in various pool sizes in traditional pooling and in the modified pooling protocol. Increases in sample Ct values from the ground truth values were calculated and plotted as ΔCt Value.

FIG. 30 provides a table for N=31 MHV-1 pooled testing qRT-PCR results.

FIG. 31 provides a table for Human COVID-19 sample pooled testing qRT-PCR results.

FIG. 32 provides a table for Compressed sensing decoded pooled testing significantly decreases the number of tests required to identify infected patients.

FIG. 33A and FIG. 33B provide compressed sensing accuracy increases with N. Random test simulation to assess the performance of compressed sensing at low and high N. A Bernoulli random matrix _{A∈{0,1}}_{^{n×N}} with Pr(Aᵢⱼ=0)=Pr(Aᵢⱼ=1)=0.5 is used for both cases. We take n=round(0.3*N), and the x is generated uniformly from _{[0,100]}_{^{N}} with sparsity round(0.05*N). The horizontal axis is the index element of x. The vertical axis is the value of the element. (A) N=10.

FIG. 34A, FIG. 34B, FIG. 34C provide representative compressed sensing decoding algorithms. FIG. 34A: Algorithm 1 virus decoding. FIG. 34B: Algorithm 2 support estimation. FIG. 34C: Algorithm 3 exhaustive search.

FIG. 35 provides adaptive request pooling matrix. Pooling matrix designed for additional testing requests. 1 indicates sample is included in the pool. 0 indicates the sample is not included in the pool.

FIG. 36 provides human COVID-19 additional testing pooling matrix. Pooling matrix designed for additional testing requests in human COVID-19 samples. N=40 (3×40). 1 indicates patient is included in the pool. 0 indicates the patient is not included in the pool.

FIG. 37 provides MHV-1 individual sample infection status after one round of testing Sample Viral Load (ng/mL)

FIG. 38 provides human COVID-19 sample second round pooling qRT-PCR results.

FIG. 39 provides human COVID-19 individual patient infection status results Sample Viral Load (ng/mL).

### DETAILED DESCRIPTION

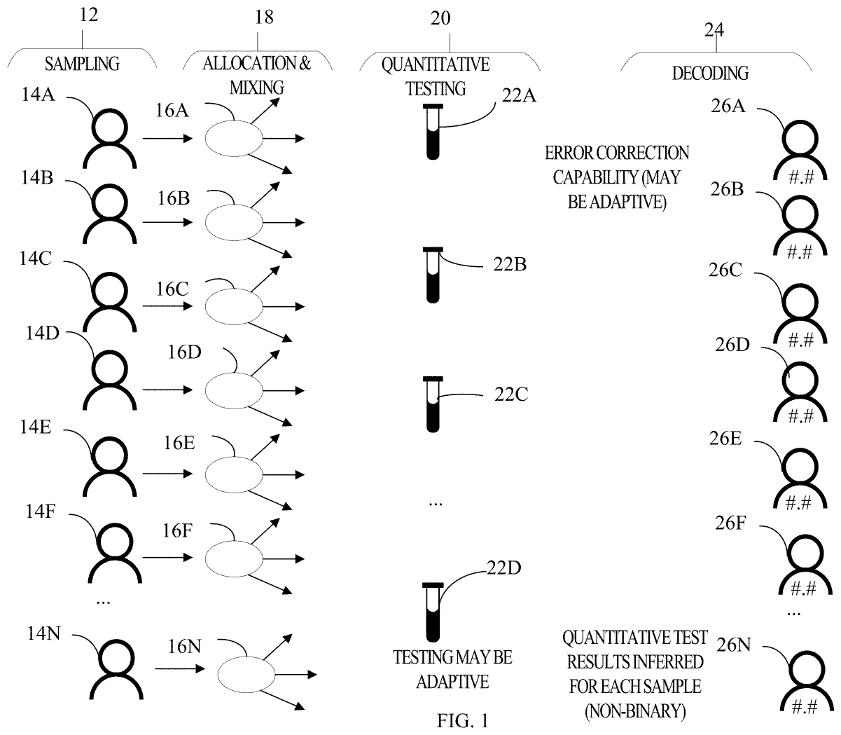

FIG. 1 is a pictorial representation providing an overview of a pooled testing method using compressed sensing. Sampling 12 is performed. As shown in FIG. 1, sampling may be performed for each of a plurality of test samples 14A, 14B, 14C, 14D, 14E, 14F, . . . , 14N. It is to be understood the test samples may be acquired from a human or other living organism, the environment (such as air samples, water samples, soil samples, rock or mineral samples, etc.), or other types of organic or inorganic compositions or materials in any number of forms or states. The present invention is not to be limited to the particular type of test sample or by or to the material being tested for (target substance) within the test sample. For purposes of illustration herein, embodiments are generally described with respect to testing for a target substance indicative of a virus, such as the COVID-19 virus, within a human. However, it is to be understood that the present invention is not to be unduly limited to this specific application.

After sampling 12 is performed, allocation and mixing 18 are performed using compressed sensing methodologies as will later be explained in more detail. The allocation and mixing defines, for each of the test samples, how much of each of the test samples is to be used (allocation) and which of the other test samples it is to be mixed with (mixing). Quantitative testing 20 is performed with pooled samples 22A, 22B, 22C, 22D. Quantitative testing is not merely a binary test (e.g. a positive or negative result) but provides for numerical results such as indication of an amount or concentration of a material being tested for within the pooled sample. Note there may be fewer pooled samples tested then individual samples due to the pooling. In each of the pooled samples 22A, 22B, 22C, 22D tested, a subset of the test samples will be included according to the defined allocations. It is to be further understood that the testing may be adaptive. That is to say, that there may be some feedback in the form of results from prior testing which is used to inform the manner in which additional allocation and mixing of test samples occurs. Adaptive testing may be advantageous in terms of minimizing the number of tests performed or to provide for error correction capability where one or more tests is not accurate.

After the test results are obtained, then decoding 24 is performed. Decoding is performed in order to infer quantitative test results (e.g. non-binary results) for each sample. The mathematics which may be used to perform the decoding will later be described herein. Generally, some of the advantages of the methods and systems described herein include the provision for quantitative (non-binary tests), quantitative estimates of the target substances for each of the test samples, the ability for error correction to improve test accuracy, the ability to use adaptive error correction to provide a certificate of accuracy, as advantages associated with particular computational algorithms for decoding.

With respect to the certification of accuracy, it is to be understood that one or more additional tests may be performed to guarantee the accuracy of results. For example, in a simple case, test samples identified as having none of the target substance may be combined and the pooled sample may be tested in a single final additional test. If the results of this final additional test indicates that there is no target substance present in the pooled sample, then the results of the tests may be certified as accurate and correct. Of course, it is contemplated that certification of accuracy may be performed in other ways by mixing selected samples for re-testing.

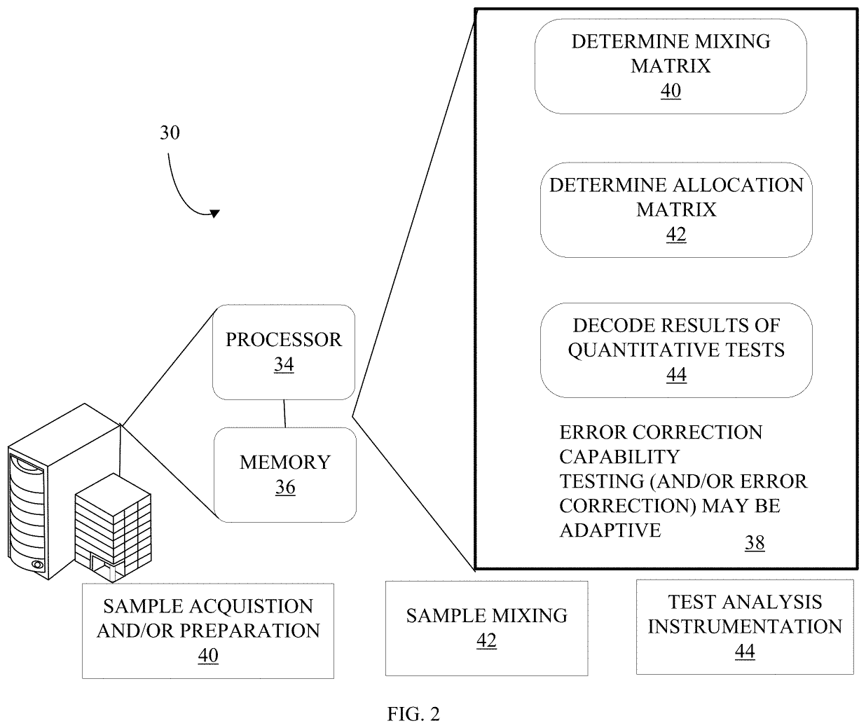

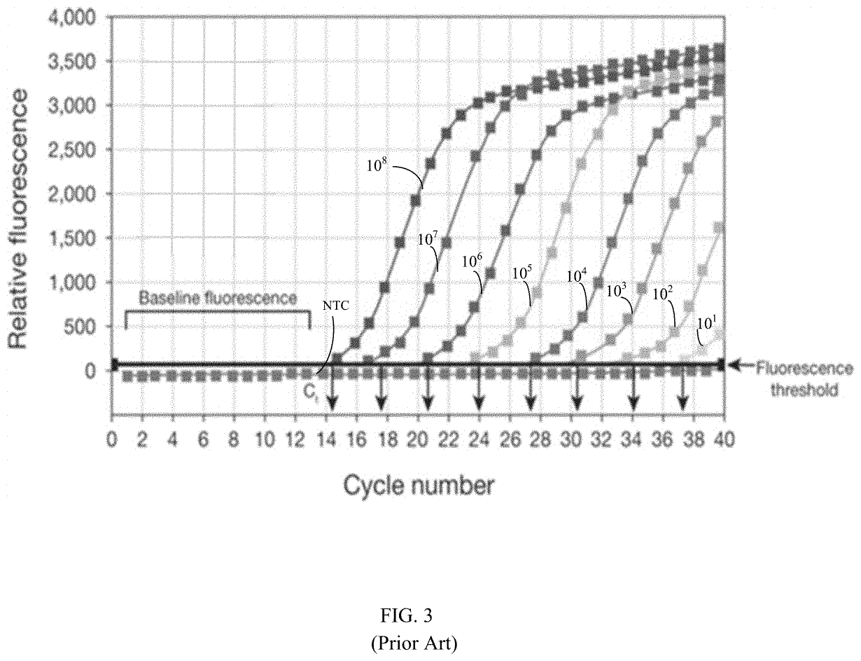

FIG. 2 is a pictorial representation of a system 30. The system 30 includes a computing device having a processor 34 and a memory 36 which may be a non-transitory machine readable memory. The memory may store a plurality of instructions for implementing a methodology. For example, the instructions may implement a method 38 to determine a mixing matrix 40 and to determine an allocation matrix 42. The mixing matrix may set forth a representation of which samples are to be pooled while the allocation matrix may set forth an amount of each of the samples to be pooled. Thus, it is to be understood, that for each test sample, portions of the test sample may be allocated to different pooled samples in different amounts. After mixing and testing, the method may decode the results of the quantitative tests 44 in order to determine quantitative estimates for a target substance in each of the individual samples. As previously explained, the methodology includes error correction capability and the testing (and/or the error correction) may be adaptive in nature. The methodology described herein may be performed with one or more modules. For example, a first module may be used for determining a mixing matrix, a second module may be used to determine an allocation matrix, and a third module may be used for decoding the results of the quantitative tests.

In addition to the computing device, other components within the system 30 may include sample acquisition and/or preparation components 40, sample mixing components 42, and test analysis instrumentation 44. The specific form of these components for sample acquisition, mixing, and analysis will depend upon the specific type of test sample and the target substance.

For purposes of explanation, PART 1 provide an additional overview of pooled sample testing using compressed sensing. PART 2 describes low-cost and high-throughput testing of COVID-19 viruses and antibodies via compressed sensing: system concepts and computational experiments. PART 3 discusses error correction codes for increasing reliability of COVID-19 virus and antibody testing through pooled testing. PART 4 concludes with additional options, variations, and alternatives. It is to be understood that different parts may use alternative nomenclature.

### Part 1: Pooled Sample Testing Using Compressed Sensing

We describe a method for increasing the throughput and reliability of diagnostic tests using the mathematical theory of compressed sensing.

Suppose that we have n test samples from n individuals in a population, and we would like to test for the presence of a substance in each individual's sample as well as determine the quantity of the substance in the sample. We use a non-negative vector x∈ [symbol] ⁿ to denote the quantities of the substance in the n samples, where xᵢ, the i-th element of x, corresponds to the quantity of target substance in the sample of the i-th individual, and [symbol] is the set of real numbers. If the i-th person is not infected xᵢ=0; if instead the i-th person is infected, xᵢ>0. If there are k<<n people affected among these n persons, x will have k positive elements, and the rest of its elements are zero. This leads to a sparse x, and we call such a vector k-sparse vector, meaning it only has at most k nonzero elements. When the vector x is sparse, compressed sensing theories offer to greatly reduce the number of testings that need to be done to accurately infer x [1] [2]. In addition, the compressed sensing method is capable of correctly recovering the x even if some number of tests produce incorrect results. In other words, the method can perform error correction. This implies high-throughput, fast, low-cost testing that is also more accurate than the naive method of testing each individual's sample separately. The basic idea of compressed sensing is to observe mixed or pooled samples of elements of x through a wide measurement matrix (as introduced below).

We first design a “mixing matrix” E of dimension m×n, where m is the number of tests we will need to run to recover x. We let each element of E be either 0 or 1. We denote the element of E in the i-th row and j-th column as E_{i,j}. If E_{i,j}=1, where 1≤i≤m and 1≤j≤n, (part of) the j-th person's sample will be mixed with samples from other persons, and this mixed sample will be tested for the target substance in the i-th test. If E_{i,j}=0, the i-th test will not involve the j-th person. The sample of the j-th person can be involved in multiple testings, the number of which is equal to the number of ‘1’s in the j-th column of E. Often we have m×n, thus making the tests more efficient and increasing the throughout of the tests.

$${E}_{i , j} = \{ \begin{matrix}1 & if sample j participates in testing i , \\ 0 & otherwise\end{matrix} \qquad ( 1 )$$

Since a person's sample is involved in multiple testings, we need to allocate a portion of that person's sample for each of the involved testings of that person. Thus for each j, 1≤j≤n, we associate the j-th person's sample with an “allocation” vector wⱼ∈ [symbol] ^{m}, whose elements are nonnegative and ∥wⱼ∥₁≤1 (the summation of wⱼ's elements are no more than 1). For example, if the i-element of wⱼ is 0.2, it means that 20 percent of the sample from the j person participates in the i-th testing.

Using wⱼ's, we can form an allocation matrix W as

W=[w₁,w₂, . . . ,wₙ]

We define the actual measurement matrix A of dimension m x n as

A=E⊙W,

where ⊙ means elementwise multiplication.

Then the generalized compressed sensing testing result vector y∈ [symbol] ^{m} is given by

y=ƒ(A×x)+v+e (2)

where each element of y represents the estimate of the target substance in a single test, ƒ(⋅): [symbol] ⁿ→ [symbol] ^{m} is a functional modeling non-linearity and randomness associated with the measurement process, v is a random noise vector and e can be a vector containing potential outliers modeling incorrect test results.

As a special case, e.g. in an ideal real-time qPCR test for viral RNA, we can have ƒ(Ax)=Ax. However, this formulation is very general, and can be used to model other types of non-linearity or randomness in testing. For example, for an end-point PCR or if we only use the real-time PCR to check for the presence of viral RNA, the functional ƒ(⋅) can output a vector of ‘true’ or ‘false’ depending on whether the quantity of RNA is above a certain significance threshold. In this document, we will use the ideal linear model as an example: ƒ(Ax)=Ax.

Compared with group testing, in our compressed sensing systems, the output y can work with real numbers or other general formats such as the whole amplification plot of qPCR, and can glean more information from each test (or measurement) than binary information. Since compressed sensing can retain more information about the vector x, in general fewer tests are needed for inferring x or the support of x. For example, compressed sensing can only use

$$m = O ( k log ( \frac{n}{k} ) )$$

tests to fully cover x, while group testing needs

$$m = O ( {k}^{2} log ( \frac{n}{k} ) ) test [ 3 ] .$$

### I. Design of Measurement Matrix A

To achieve robust and rapid testing, we design the matrix A in the following ways.

(1) Recall that we have the measurement matrix A=E⊙W, where E is the mixing matrix and W is the allocation matrix. The matrix E is a 0-1 matrix with ‘0’ or ‘1’ elements. The number of ‘1”s in the matrix E should be small, thus making matrix E a sparse matrix. This is because we would like the number of ‘1's in E to be as small as possible in order to minimize the complexity of mixing samples from different persons, and minimize the probability of mistakes in mixing. For each column of E, we also consider constraining the number of ‘1”s. This is because we do not want to dilute the quantity of the j-th person's sample too much by distributing it to too many tests. If it is distributed to too many tests, the quantity from the j-th person for each individual test can be too little for going above the detection threshold of the test machines.

All these physical constraints and considerations motivate us to propose using sparse bipartite graph measurement matrices for the design of E and A. In particular, we propose to use the expander-graph based compressed sensing, which was proposed for general compressed sensing [4][5]. The expander graph-based measurement matrix is a 0-1 matrix derived from expander bipartite graphs. It comes with efficient decoding algorithms and provable performance guarantees for testing. Moreover, the number of ‘1’s in each column can be upper bounded for the expander graph based matrices, which complies with the physical constraint that a person's sample cannot be distributed to too many samples.

(2) We have the freedom of designing the allocation matrix, but, for simplest presentations, we can choose the simplest allocation design of evenly dividing the sample into the measurements involved. Namely, A will be obtained by dividing each column of E by the total number of ‘1’s in that column. It is entirely possible to use other allocation matrices for better performance or more efficient decoding.

II. Detection (Decoding) Algorithms from Compressed Mixed Measurements

Due to the extensive developments of compressed sensing [1][2] over the last two decades, there are many decoding algorithms to infer x from y, such as basis pursuit ( [symbol] minimization), LASSO, message passing style algorithms [4] [6], and greedy algorithms such as orthogonal matching pursuits. One can potentially choose any of these algorithms to do the decoding. We also notice that the signal x is non-negative, which can be used to boost the efficiency of compressed sensing [7].

However, many of the algorithms from the literature have performance guarantees or good empirical performance when the dimensions of A are very large, or m is asymptotically proportional to n when n goes to infinity. In practice, some of these algorithms can experience severe performance degradation for finite n corresponding to practical applications such as virus testing. We focus on developing fast algorithms for realistic population sizes n.

We start with the iterative algorithms for expander graphs [4]:

(1) [symbol] minimization.

This is equivalent to exhaustive search over all the possible sets of k persons and then solve for x using an overdetermined system for each of these sets using y. Formally, if there is no noise in the observation, we are solving

$$\begin{matrix}minimize {‖ x ‖}_{0} \\ subject to y = Ax , & ( 3 ) \\ x \geq 0. & ( 4 )\end{matrix}$$

where ∥x∥₀ is the number of non-zero elements in vector x. The [symbol] minimization is an NP-hard problem. But the exhaustive search or its modifications might still be a good choice for certain applications if the population size is small enough to make it computationally feasible, since it gives great performance in minimizing false positive and false negative rates.

(2) [symbol] minimization.

To reduce the computational complexity, we can often relax (4) to its closest convex approximation—the [symbol] minimization problem:

$$\begin{matrix}minimize {‖ x ‖}_{1} \\ subject to y = Ax , & ( 5 ) \\ x \geq 0. & ( 6 )\end{matrix}$$

where ∥x∥₀ is the sum of the absolute values of all the elements in x. The optimization problems in (4) and (6) enforce the constraint y=Ax which does not allow for testing errors. If we relax this assumption and allow that the vector e in (2) may be non-zero but sparse (i.e. a small proportion of the test results may be in error), we can derive a more flexible optimization problem:

minimize ∥x∥₁+λ∥y−Ax−v∥₁ (7)

subject to y=Ax, (8)

∥v∥₂≤ν, (9)

x≥0, (10)

where the constraint (9) comes from the assumption that residual measurement noise is small, once we account for the small number of incorrect test results modeled by the sparse vector e. The parameter λ in (7) can be used to tradeoff test throughput for greater tolerance of test errors i.e. by tuning this parameter, we may be able to increase the accuracy of each individual's diagnosis even if many tests are in error by simply increasing the number of tests.

After solving for the vector x, we can set a threshold τ>0 such that if xⱼ≥τ, we declare the test is positive for the j-th person; otherwise, we declare the testing result as negative.

It has been shown that the optimal solution of [symbol] minimization can be obtained by solving [symbol] minimization under certain conditions (e.g. Restricted Isometry Property or RIP [1][8] [2][9][10]. A necessary and sufficient condition under which a vector x with no more than k nonzero elements can be uniquely obtained via [symbol] minimization is Null Space Condition (NSC), for example, see [11], [12]. While the RIP condition and NSC condition are normally satisfied for large-dimension matrix A, there are algorithms which can precisely verify the null space condition for small-size problems, which will be especially useful for designing optimal pooling strategies or the compressed sensing matrices [13][14] [15].

### III. Adaptive Compressed Sensing-Based Testing

The formulations (4), (6) and (10) are all non-adaptive designs i.e. these are all methods where the sample mixtures are all prepared ahead of time before any tests are conducted. A more flexible and powerful variant of pooled testing methods are adaptive tests, where we are able to design sample mixtures in real-time taking into account the results of tests on previous sample mixtures.

Our proposed adaptive testing method is motivated by adaptive error correction procedures called Automatic Repeat Request (ARQ) commonly used in communication networks. In the adaptive compressed sensing method, the measurement matrix A=E⊙W will not be determined fully in advance. Instead, we will start with only the first few rows r<<m of the matrix A. Once the first r tests have been performed and the corresponding results are available, we attempt to recover the diagnosis vector x. If the r is small, there is a good chance that the vector x is under-determined by the minimal number of test results available so far. An important detail here is that since the vector x is non-negative and sparse, it is very easy to check if a tentative estimate is correct: just prepare a set of mixed samples containing non-zero portions of the samples from each individual that was identified as being infection-free. All of these tests must result in negative test results. Any positive test results show that our estimate of x is inaccurate, but now we have more test results which can be used to refine the estimate. We continue in this fashion until our estimated test results are confirmed as accurate. At the end of the adaptive procedure, we will have an estimate of the infection vector x along with a certificate of accuracy.

### IV. Features

Group testing [16] is a well-known method that has become widely accepted for infectious disease diagnostic testing [17], [18] as well as for other applications such as DNA hybridization [19] and genome data processing [20]. The relationship between group testing, compressed sensing and information theory [21] are also well-known. Some of the advantageous features of the methods and systems described herein are as follows.
Non-binary tests. Most work on traditional group testing are based on binary diagnostic tests that simply look for the presence or absence of the target substance in the test sample. Our method uses quantitative tests that provide an estimate of the amount of target substance contained in the test sample. This is richer information and allows our method to do better than group testing. Indeed group testing is a simple special case of our method.
Quantitative estimates of target substance. Our method also produces quantitative estimates of the amount of the target substance found in each individual's sample rather than just a positive/negative diagnosis. For a virus test, our method can provide an estimate of the viral load of each person tested rather than just presence or absence of the virus. This may be useful medical information.
Error correction capability. Traditional group testing has been mostly focused on minimizing the number of tests; group testing does not provide any way of reducing testing errors. Our method uses the error correcting capability of pooled testing to actually increase the diagnostic accuracy of test results without performing more tests. This is a powerful new capability that has no counterpart in traditional group testing.
Adaptive error correction. Adaptive algorithms for compressed sensing are not new [22]. Our proposed adaptive method takes advantage of the non-negativity of the vector x in a novel way. Also, traditional adaptive sensing is focused on increasing the efficiency of the sensing i.e. minimizing the number of required tests. We add a novel feature which is providing a certificate of accuracy of the final test results.
Novel computational algorithms for decoding. Our decoding algorithms for processing the pooled sample test results are novel. Our algorithms perform well for finite population sizes for which classical methods from the compressed sensing literature that are designed for very large data sets often show poor performance. We are also able to use machine learning to optimize the decoding process.

### Part 2: Low-Cost and High-Throughput Testing of COVID-19 Viruses and Antibodies Via Compressed Sensing: System Concepts and Computational Experiments

Coronavirus disease 2019 (COVID-19) is an ongoing pandemic infectious disease outbreak that has significantly harmed and threatened the health and lives of millions or even billions of people. COVID-19 has also negatively impacted the social and economic activities of many countries significantly. With no approved vaccine available at this moment, extensive testing of COVID-19 viruses in people are essential for disease diagnosis, virus spread confinement, contact tracing, and determining right conditions for people to return to normal economic activities. Identifying people who have antibodies for COVID-19 can also help select persons who are suitable for undertaking certain essential activities or returning to workforce. However, the throughputs of current testing technologies for COVID-19 viruses and antibodies are often quite limited, which are not sufficient for dealing with COVID-19 viruses anticipated fast oscillating waves of spread affecting a significant portion of the earth's population.

Here, we propose to use compressed sensing (group testing can be seen as a special case of compressed sensing when it is applied to COVID-19 detection) to achieve high-throughput rapid testing of COVID-19 viruses and antibodies, which can potentially provide tens or even more folds of speedup compared with current testing technologies. The proposed compressed sensing system for high-throughput testing can utilize expander graph based compressed sensing matrices developed by us [4].

### 1 Introduction

The ongoing Covid-19 pandemic has already claimed thousands of human lives. In addition, it has also forced a worldwide shutdown of social life and commerce, and the resulting economic depression has caused tremendous suffering for millions of people.

In the absence of a vaccine, the experience of public health authorities in several countries has shown that large-scale shutdowns can only be safely ended if a systematic “test and trace” program [32, 35] is put in place to control the spread of the virus. This, in turn, is predicated on the widespread availability of mass diagnostic testing. However, most countries including the US are currently experiencing a scarcity [34] of various medical resources including tests [25].

One simple method to increase the effective testing capacity by testing pooled samples of several test subjects collectively instead of testing samples from each person individually. This idea of “group testing” goes back many decades [16] and is based on the following intuition. If the rate of infection in the population is relatively low, statistically, most individual will test negative. With group testing, a single negative test result on a pooled sample immediately shows that all individuals in that pool are infection-free.

This potentially allows us to reduce the total number of tests per subject so the throughput of the existing testing infrastructure is increased [27] i.e. a much larger number of people can be tested compared to individual testing while keeping the number of tests the same.

Pooling does have its risks. The additional pre-processing required for preparing the pooled samples could affect the accuracy of the test because of possible degradation or contamination of the RNA. Pooling also requires dilution of the individual samples, and this in turn may increase the chances of a false negative result. However, pooling tests have been successfully used for diagnostic testing for infectious diseases in the past [18, 17]. Preliminary studies on the Covid-19 virus also show that pooling samples [41] can be effective with existing tests.

The current testing bottlenecks in the Covid-19 crisis has led to a resurgence of interest in using group testing methods for Covid-19 diagnosis. Specifically, there have been recent studies [40, 38, 28] into adapting pooling methods similar to [16] for Covid-19 testing. In [42], the authors studied noisy group testing for virus detection.

Here, we propose a different approach based on the compressed sensing theory [23] [25][2] for detection of viruses and antibodies using pooled sample testing. In compressed sensing, the measurement reading is not just a binary reading (‘positive’ or ‘negative’) as in group testing, but instead the measurement reading of compressed sensing can be real-numbered quantification of the quantity of target DNA in the pooled sample. The traditional group testing methods such as [16] can be thought of as special cases of the more powerful compressed sensing framework proposed herein. This is because the measurement reading of group testing is a binary reduction of the real-numbered quantification of compressed sensing. Through compressed sensing, it is possible to test n persons for viruses by only using O(k log(n)) tests, where k is the number of virus-infected persons. This is a significant reduction compared with testing each individual person, which would require n testing. This can translate into an increase of test throughput in the order of n/(k log(n)), which can be quite significant if the number of infected people is much smaller than the total population.

Indeed, the real-numbered quantification from compressed sensing can greatly help speed up the testing of viruses and reduce the cost of testing, by taking advantage of the sparsity of virus infections in the population. Compared with conventional group testing (including non-adaptive and adaptive group testing), compressed sensing has the following advantages:
(1) Compressed sensing uses real-numbered quantitative measurement results (quantification of target DNA etc.) to infer virus infections or antibodies. These measurement readings contain more information about the collected samples than the binary readings of group testing. This will make inference from compressed sensing measurements more robust against noises an outlier in the measurements and require fewer tests.
(2) Compressed sensing is known to require fewer measurements (or lower sample complexity) to infer virus infections than group testing. The sparsity k that compressed sensing can handle for successful detection is allowed to grow linearly (proportionally) with n, while the recoverable sparsity k is of the order O\sqrt{n} for non-adaptive group testing [3]. This will potentially translate higher testing throughput for compressed sensing than group testing.
(3) The inference results from compressed sensing not only reveal which persons test positive or negative, but also reveal a quantitative evaluation of infections for the persons who test positive. For example, it can reveal the viral loads (copies/ml) of persons who test positive. These quantitative results can help achieve better diagnosis and treatment of infected persons and can also help study infectious power of viruses in different phases of infections.

There are broadly two types of tests for Covid-19: (a) serological tests that look for the presence of antibodies to the virus, or (b) swab tests that look for RNA from the live virus. While antibody tests have certain advantages e.g. can detect infections even after the subject has recovered, the most common tests currently used in the US and recommended by the CDC are swab tests. These tests use the Reverse Transcription Polymerase Chain Reaction (RT-PCR) process to selectively amplify DNA strands produced by viral RNA specific to the Covid-19 virus.

The RT-qPCR process which is considered the gold standard for the detection of mRNA consists of three distinct steps: (1) reverse transcription of RNA into cDNA, (2) selective amplification of a target DNA fragment using the Polymerase Chain Reaction (PCR), and (3) detection of the amplification product. While the simple “end-point” version of PCR only allows binary detection (presence or absence) of a target RNA sequence, the real-time or quantitative version of the PCR process (qPCR) [26] also allows the quantification of the RNA i.e. it produces an estimate of the quantity of the RNA material present in the sample [33].

Some researchers [31] have proposed the Reverse Transcription Loop-Mediated Isothermal Amplification (RT-LAMP) as a potentially cheaper and faster alternative to RT-PCR for swab tests. While we focus on tests based on the RT-qPCR process, the methods proposed are also compatible with RT-LAMP [36] and other DNA amplification methods.

Here, we propose to use compressed sensing to detect viruses and antibodies of COVID-19. Considering the physical and complexity constraints of pooling for compressed sensing, we identify sparse bipartite graph based measurement matrices for compressed sensing applied to this purpose. In particular, we propose to use expander graph based measurement matrices [4] for pooling or measurement designs.

As mentioned above, group testing has a long history of being used to detect pathogens, tracing back to World War II, and it has also recently been applied to testing COVID-19 viruses [40, 38]. To the best of our knowledge, this work might be the first to develop compressed sensing techniques for detecting viruses using qPCR and other tools, especially when applied to COVID-19 viruses. On a related but different subject, we note that compressed sensing was proposed in [39] to study human genetics, and used to identity people with rare alleles (“allele is one of two or more alternative forms of a gene that arise by mutation and are found at the same place on a chromosome.”).

### 2. Compressed Sensing for High-Throughput Virus Detection: System Model and Problem Formulation

In this section, we describe the system architecture of using compressed sensing to speed up the testing of COVID-19 viruses or antibodies, including sensing matrix design and decoding algorithm design. We will focus on developing such systems using Polymerase Chain Reaction (PCR) machines, especially real-time PCR (quantitative PCR, qPCR or RT-PCR) machines, to test the viruses, though the concepts and ideas introduced herein extend to testing viruses using other technologies or platforms and also to testing antibodies. (We note that in the literature, there are inconsistencies about the meanings of “RT-PCR”, which are used as abbreviations for both reverse transcription PCR and real-time PCR.) We start by introducing some background knowledge on the real-time quantitative PCR [37].

The polymerase chain reaction (PCR) is one of the most powerful and widely used technologies in molecular biology to detect and quantify specific sequences within a DNA or cDNA template. Using PCR, specific sequences within a DNA or cDNA template can be copied, or amplified, to thousands or to a million times using sequence-specific oligonucleotides, heat-stable DNA polymerase, and thermal cycling [30]. PCR theoretically amplifies DNA exponentially, doubling the number of target molecules with each amplification cycle.

To address the need of robust quantification of DNA, real-time polymerase chain reaction (real time PCR) was developed based on the polymerase chain reaction (PCR). Real-time PCR is carried out in a thermal cycler (providing temperature conditions for each cycle of reactions), but with the capacity to illuminate each sample with a beam of light and detect the fluorescence emitted by the excited fluorophore [37].

In traditional (endpoint) PCR, detection and quantification of the amplified sequence are performed at the end of the reaction after the last PCR cycle. In real-time quantitative PCR, PCR product (the amplified sequences) is measured at each PCR cycle. Namely, Real-time PCR can monitor the amplification of a targeted DNA module in the PCR in real time. By monitoring reactions during the exponential amplification phase of the reaction, users can determine the initial quantity of the target with great precision. The working physical principle of the RT-PCR is that it detects amplification of DNA in real time by the use of fluorescent reporter. The fluorescent reporter signal strength is directly proportional to the number of amplified DNA molecules.

Real-time PCR commonly relies on plotting fluorescence against the number of cycles on a logarithmic scale to perform DNA quantification. During the exponential amplification phase, the quantity of the target DNA template (amplicon) doubles every cycle. A threshold for detection of DNA-based fluorescence is set 35 times of the standard deviation of the signal noise above background. The number of cycles at which the fluorescence exceeds the threshold is called the threshold cycle (Cₜ) or, quantification cycle (C_{q}). One can then use this threshold cycle Cₜ to determine the quantity of target DNA in the sample. In ideal cases, if the threshold cycle of a DNA sample A precedes that of another sample B by N cycles, then this DNA sample A contains 2^{N} times more target DNAs than DNA sample B at the beginning of the reaction. In practice, people often use the standard curve method for real-time PCR to determine the relation between threshold cycle Cₜ and target quantity.

### 2.1 Compressed Sensing System for High-Throughput Rapid Testing

In this subsection, we propose and describe a compressed sensing system to perform high-throughput rapid testing of COVID-19 and antibodies. We remark that this system also applies to testing of other types of viruses or antibodies.

Suppose that we have collected n samples of n persons, and we would like to test how many among them have viruses and what quantity of viruses they have. (It is also possible that we can collect more than 1 sample from a person, but for simplicity of presentations, we stick with 1 sample per person.) We use a non-negative vector x∈ [symbol] ⁿ to denote the quantities of COVID-19 viruses in the samples of these n persons, where xᵢ, the i-th element of x, corresponds to the quantity of target DNA in the sample of the i-th person, and [symbol] is the set of real numbers. If the i-th person is not infected or has no COVID-19 virus, xᵢ=0 or very close to 0; if instead the i-th person is infected, xᵢ>0. If there are k (k can be small compared with n) people affected among these n persons, x will have k positive elements, and the rest of its elements are zero. This leads to a sparse x, and we call such a vector k-sparse vector, meaning it only has k nonzero elements. When the vector x is sparse, compressed sensing theories offer to greatly reduce the number of testings that need to be done to accurately infer x [25] [2]. This implies high-throughput, fast and low-cost testing for detecting viruses. The basic idea of compressed sensing is to observe mixed or pooled samples of elements of x through a wide measurement matrix (as introduced below). Compared with group testing, compressed sensing can correctly infer the real-numbered values of x (which will be useful for research of different phases of infections, better diagnosis, treatment of infected persons), requires fewer testing to detect positive cases, and is more robust against noisy observations.

We then design mixing matrix E of dimension m×n, where m can be significantly smaller than n. In fact, m is the number of tests we will eventually need to run to detect viruses, and often we have m<<n, thus making the tests more efficient and increasing the throughout.

$${E}_{i , j} = \{ \begin{matrix}1 if sample j participates in testing i , \\ 0 otherwise\end{matrix}$$

Namely, if E_{i,j}=1, where 1≤i≤m and 1≤j≤n, (part of) the j-th person's biological sample will be mixed with samples from other persons, and we will perform PCR (or other testing technologies) over this mixed sample in the i-th test. Otherwise, the i-th test will not involve the j-th person. The sample of the j-th person can be involved in multiple testings, the number of which is equal to the number of ‘1’s in the j-th column of E.

Since a person's sample is involved in multiple testings, we need to allocate a portion of that person's sample for each of the involved testings of that person. Thus for each j, 1≤j≤n, we associate the j-th person's sample with an “allocation” vector wⱼ∈ [symbol] ^{m}, whose elements are nonnegative and ∥wⱼ∥₁≤1 (the summation of wⱼ's elements are no more than 1). For example, if the i-element of wⱼ is 0.2, it means that 20 percent of the sample from the j person participates in the i-th testing.

Using wⱼ′s, we can form an allocation matrix Was

W=[w₁,w₂, . . . ,wₙ].

We define the actual measurement matrix A of dimension m×n as

A=E⊙W,

where 0 means elementwise multiplication.

Then the generalized compressed sensing testing result vector y∈ [symbol] ⁿ is given by

y=ƒ(A×x)+v+e,

where each element of y represents the measurement results of the DNA quantity in a single test (as can be computed by looking at the threshold cycle Cₜ's value), ƒ(⋅): [symbol] ⁿ× [symbol] ^{m} is a functional modeling non-linearity and randomness associated with the measurement process, v is a random noise vector and e can be a vector containing potential outliers.

As a special case, in an ideal real-time PCR, we can have ƒ(Ax)=Ax. However, this formulation is very general, and can be used to model other types of non-linearity or randomness in testing. For example, for a traditional end-point PCR or if we only use the real-time PCR to see whether viruses exist, the functional ƒ(⋅) can output a vector of ‘true’ or ‘false’ depending on whether the quantity of DNA samples is above a certain significance threshold. Herein, we focus on the RT-PCR, and assume it is ideal in the sense that the quantity of DNA sample inferred from its readings is ƒ(Ax)=Ax. Compared with group testing, in our compressed sensing systems, the output y can work with real numbers or other general formats such as the whole amplification plot of qPCR, and can glean more information from each test (or measurement) than binary information. Since compressed sensing can retain more information about the vector x, in general fewer tests are needed for inferring x or the support of x. For example, compressed sensing can only use

$$m = O ( k log ( \frac{n}{k} ) )$$

tests to fully recover x, while group testing needs

$$m = O ( {k}^{2} log ( \frac{n}{k} ) )$$

tests [12].

### 2.2 Design of Measurement Matrix A

To achieve robust and rapid testing, we design the matrix A in the following ways.
(1) Recall that we have the measurement matrix A=E⊙W, where E is the mixing matrix and W is the allocation matrix. The matrix E is a 0-1 matrix with ‘0’ or ‘1’ elements. The number of ‘1”s in the matrix E should be small, thus making matrix E a sparse matrix. This is because we would like the number of ‘1’s in E to be as small as possible in order to minimize the complexity of mixing samples from different persons and minimize the probability of mistakes in mixing. For each column of E, we also consider constraining the number of “1”. This is because we do not want to dilute the quantity of the j-th person's sample too much by distributing it to too many tests. If it is distributed to too many tests, the quantity from the j-th person for each individual test can be too little for going above the detection threshold of the PCR machines.

All these physical constraints and considerations motivate us to propose using sparse bipartite graph measurement matrices for the design of E and A. In particular, we propose to use the expander-graph based compressed sensing, which was proposed for general compressed sensing [4][5]. The expander graph-based measurement matrix is a 0-1 matrix derived from expander bipartite graphs. It comes with efficient decoding algorithms and provable performance guarantees for testing. Moreover, the number of ‘1’s in each column can be upper bounded for the expander graph-based matrices, which complies with the physical constraint that a person's sample cannot be distributed to too many samples.
(2) We have the freedom of designing the allocation matrix, but, for simplest presentations, we can choose the simplest allocation design of evenly dividing the sample into the measurements involved. Namely, A will be obtained by dividing each column of E by the total number of 1 in that column. It is entirely possible to use other allocation matrices for better performance or more efficient decoding.
(3) Considering physical and operational constraints, matrix A cannot be too wide and too tall at the same time.
   2.3 Detection (Decoding) Algorithms from Compressed Mixed Measurements

From the measurement result y, one can infer the quantity of DNA sample (or viruses) associated with each person. Due to the extensive developments of compressed sensing [25][2] over the last two decades, there are many decoding algorithms to infer x from y, such as basis pursuit ( [symbol] minimization), LASSO, message passing style algorithms [4][6], and greedy algorithms such as orthogonal matching pursuits. One can potentially choose any of these algorithms to do the decoding. We also notice that the signal x is nonnegative, which can be used to boost the efficiency of compressed sensing [7].

However, for detecting viruses or antibodies, we still need to choose or develop fast and robust decoding algorithms in this particular application. The reason is that many of the aforementioned algorithms have performance guarantees or good empirical performance when the dimensions of A are very large, or m is asymptotically proportional to n when n goes to infinity. This is not the case for compressed sensing for virus detection, since we have a measurement matrix of finite and possible very limited sizes. Some of these algorithms can experience severe performance degradation because of size limitations of A.

Because of the limited sizes of matrix, A, and to reduce the false positive rate and false negative rates of the testing, we can start with the following two algorithms, and the message passing style iterative algorithms for expander graphs [4]:
(1) [symbol] minimization.

This is equivalent to exhaustive search over all the possible sets of k persons with viruses and then solve for x using an overdetermined system for each of these sets using y. Formally, if there is no noise in the observation, we are solving

minimize ∥x∥₀

subject to y=Ax; (2)

x≥0: (3)

where ∥x∥₀ is the number of non-zero elements in vector x. The [symbol] minimization is an NP-hard problem. But the exhaustive search or its modifications might be good choice for this application, since it gives great performance in minimizing false positive rate and false negative rate. Since the problem of size of this application may not be big due to physical constraints, it can be computationally feasible.
(2) [symbol] minimization

To reduce the computational complexity, we can often relax (2) to its closest convex approximation—the [symbol] minimization problem:

minimize ∥x∥₁

subject to y=Ax; (2)

x≥0: (3)

where ∥x∥₁ is the sum of the absolute values of all the elements in x.

After solving for the vector x, we can set a threshold τ>0 such that if xⱼ≤τ, we declare the test is positive for the j-th person; otherwise, we declare the testing result as negative.

It has been shown that the optimal solution of [symbol] minimization can be obtained by solving [symbol] minimization under certain conditions (e.g. Restricted Isometry Property or RIP) [1][8] [2][9][10]. A necessary and sufficient condition under which a vector x with no more than k nonzero elements can be uniquely obtained via [symbol] minimization is Null Space Condition (NSC), for example, see [11][12]. While the RIP condition and NSC condition are normally satisfied for large-dimension matrix A, there are algorithms which can precisely verify the null space condition for small-size problems, which will be especially useful for designing optimal pooling strategies or the compressed sensing matrices for detection of viruses. [29][14] [15].

### 3 Numerical Experiments

In the experiments, we consider two types of binary pooling matrix: Bernoulli random matrix where each entry of the matrix is ‘0’ with probability 0.5, and is ‘1’ with probability 0.5, and measurement matrix obtained from an expander graph [4] where each column has a fixed number of ones. Experimenting with random Bernoulli pooling matrices can show the typical

performance of such pooling matrices. In practice, one needs to work with deterministic pooling matrices. To design a deterministic matrix, one can use algorithms in [15] to precisely verify the performance guarantee of a randomly generated matrix for virus testing. After the verification, we can then use it as a deterministic pooling matrix in practice.

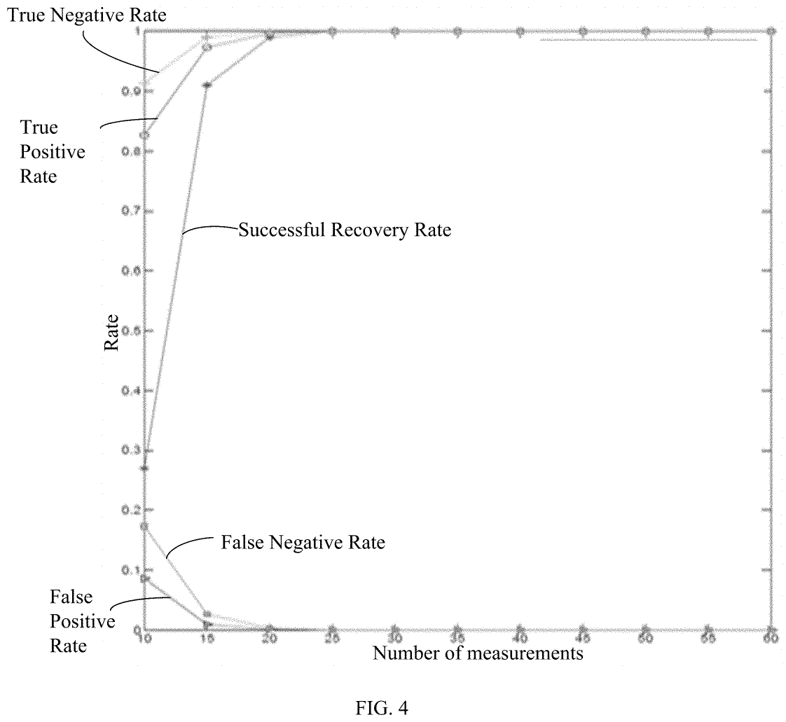

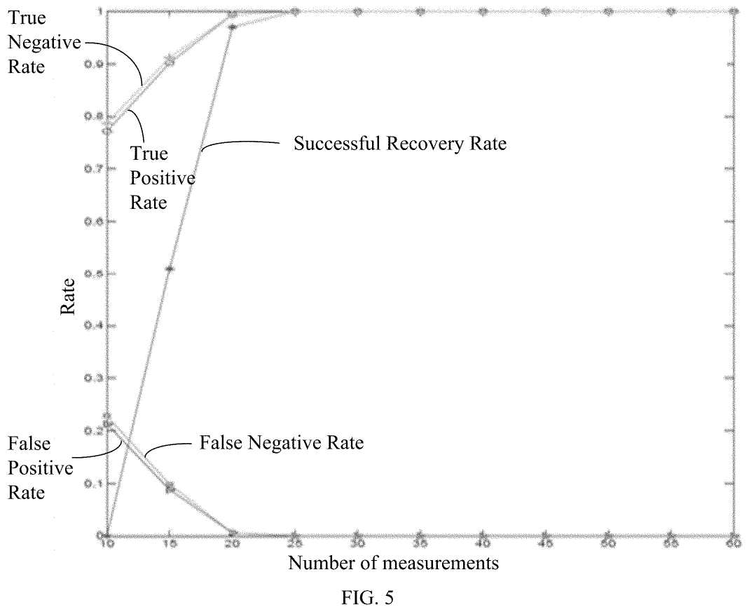

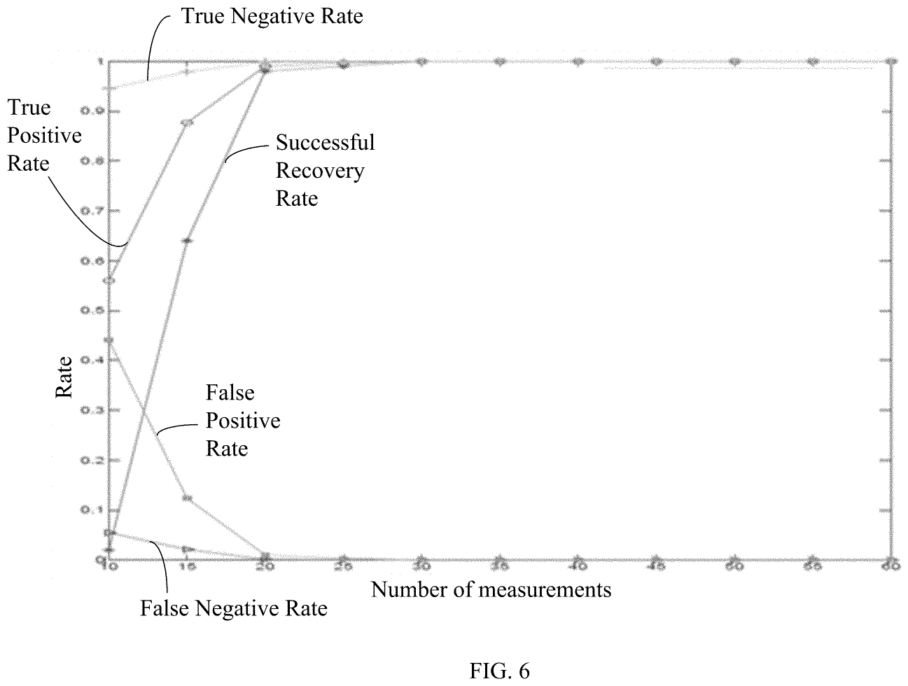

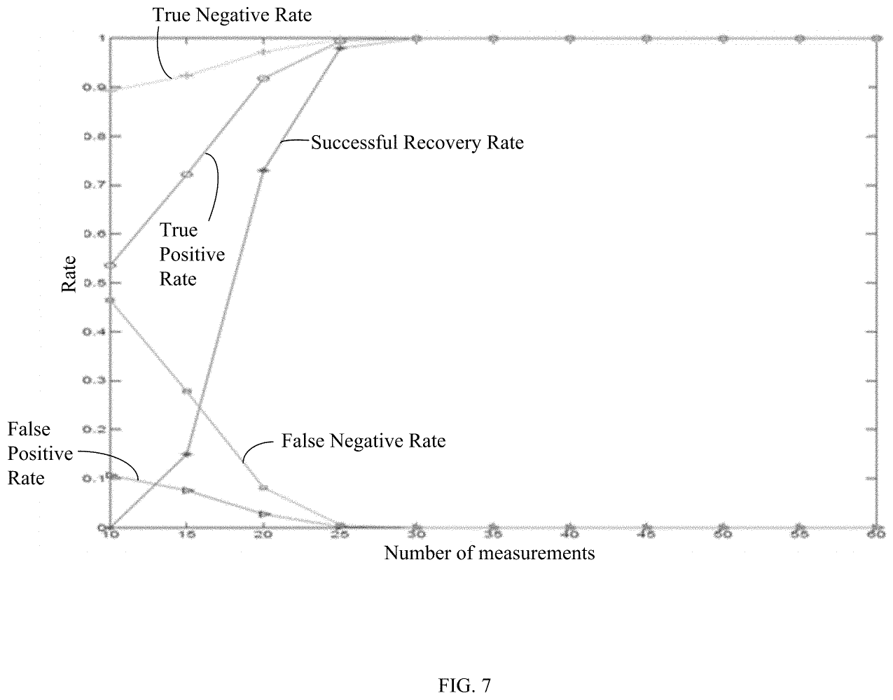

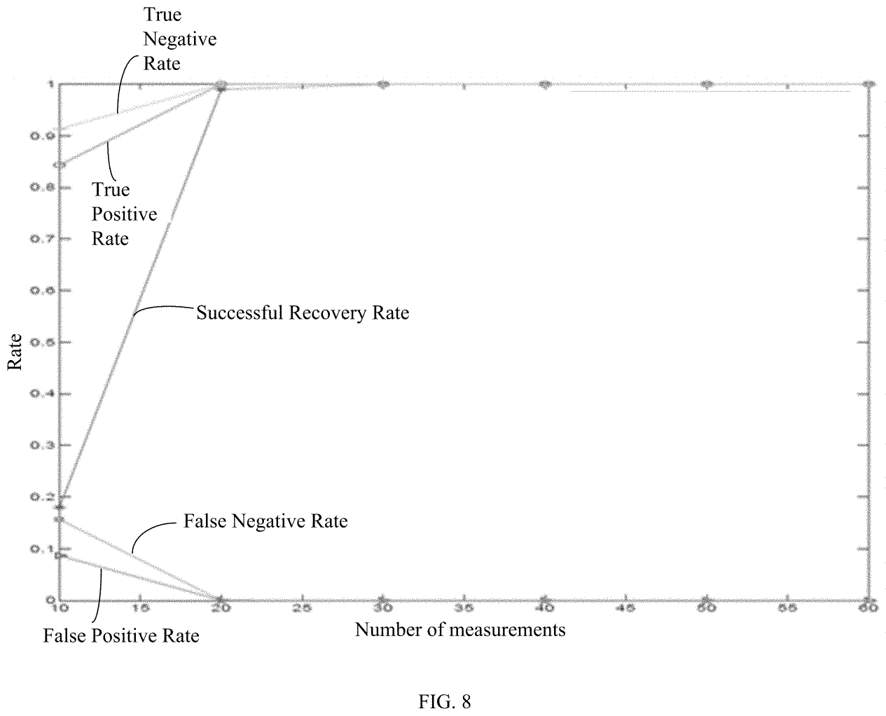

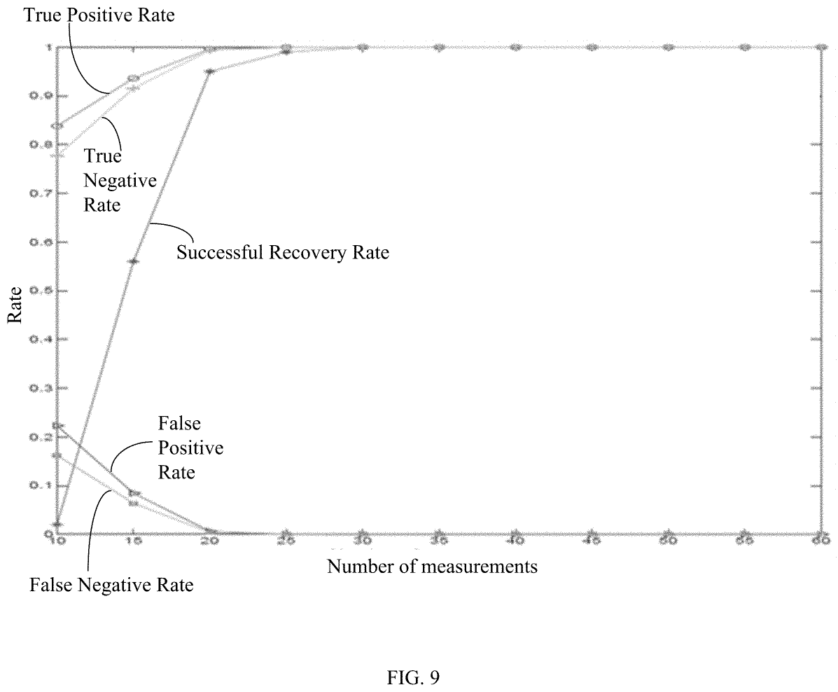

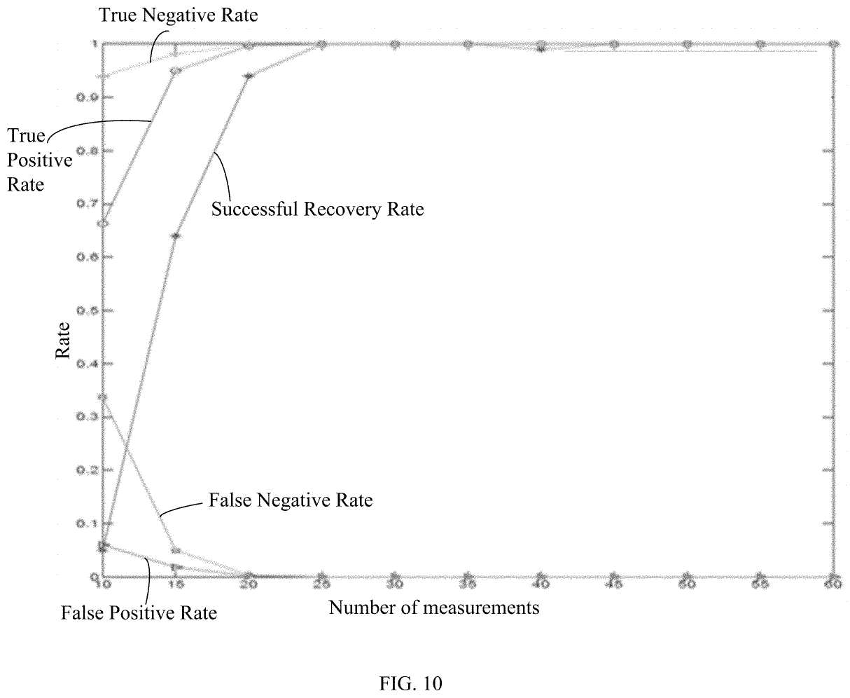

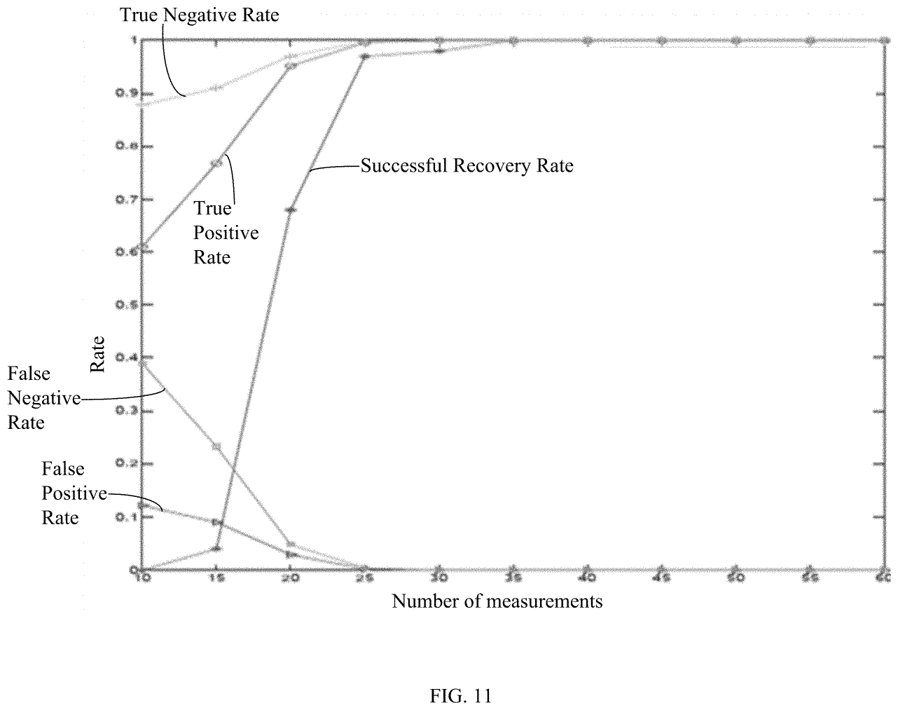

For these two types of binary pooling matrix, we consider two different values for the number of people tested, i.e., n=120 and n=60. For each of the two values of length n, we recover the value of x with different sparsity (sparsity is the number of people infected in this group of people), i.e., k=3 and k=5. In the experiments, we set random k entries of the signal of length n to be random numbers within [15][2], while the other entries are set to be positive numbers close to 0. When n=60, for each k and measurement matrix type, we take different measurements m=10, 15, 20, . . . , and 60. For each possible m, we run 100 trials to evaluate the successful recovery rate via solving

$${min}_{x ∈ {n}^{}} {‖ x ‖}_{1} , \\ s . t . Ax = y , x \geq 0 \qquad ( 6 )$$

where A∈ [symbol] ^{m×n} is the measurement matrix, x is the signal to be recovered, and y∈ [symbol] ^{m} is the measurement vector. After a signal is decoded, we use a thresholding technique to identify the persons with viruses. For each trial, we set a threshold τ=0.5. The signal entry will be determined to be ‘positive’ with viruses, if the recovered value is at least τ, and ‘negative’ if it is less than τ. We then calculate the true positive rate (TPR), true negative rate (TNR), false positive rate (FPR), and false negative rate (DNR). We also consider the recovery success rate: if the reconstruction error (the Euclidean distance between the true signal x and the recovered signal \hat{x}) is smaller than 10⁻³, we count the recovery as a success. The numerical results are shown in FIG. 4 to FIG. 7 for Bernoulli measurement matrices. Numerical results are shown in FIGS. 8 to 11 for expander graph-based measurement matrices. As we can see from these figures, n=60, we only need around m=20 tests to achieve very low false negative rates and false positive rates, which means that we can increase the throughput of virus testing by

$$\frac{n}{m} \approx 3$$

times. For n=120, we also need around m=20 tests to achieve low false negative and false positive rates, which translates to around

$$\frac{n}{m} \approx 6$$

times increase in test throughput. For k=2 and n=200, when we use expander graph based pooling matrix with 5 ‘1’s in each column, we can already achieve a near zero false positive and false negative rates when m=20. This translates to a

$$\frac{200}{20}$$

folds of speedup in test throughput.

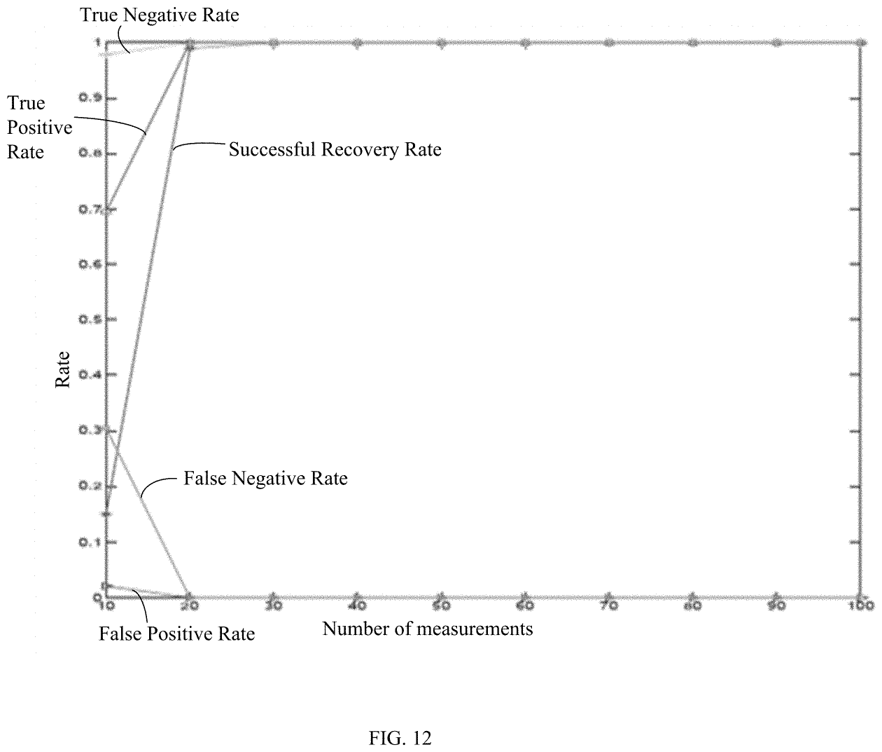

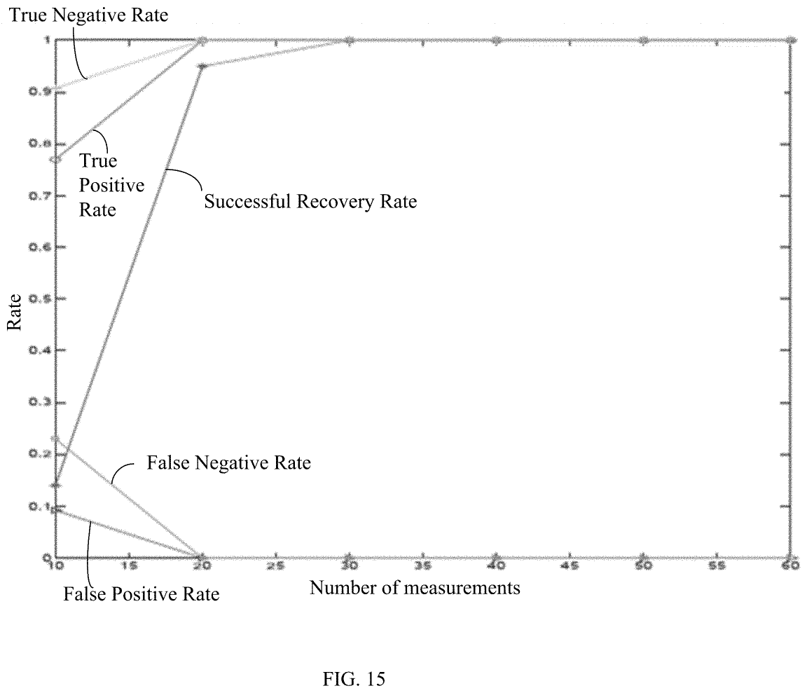

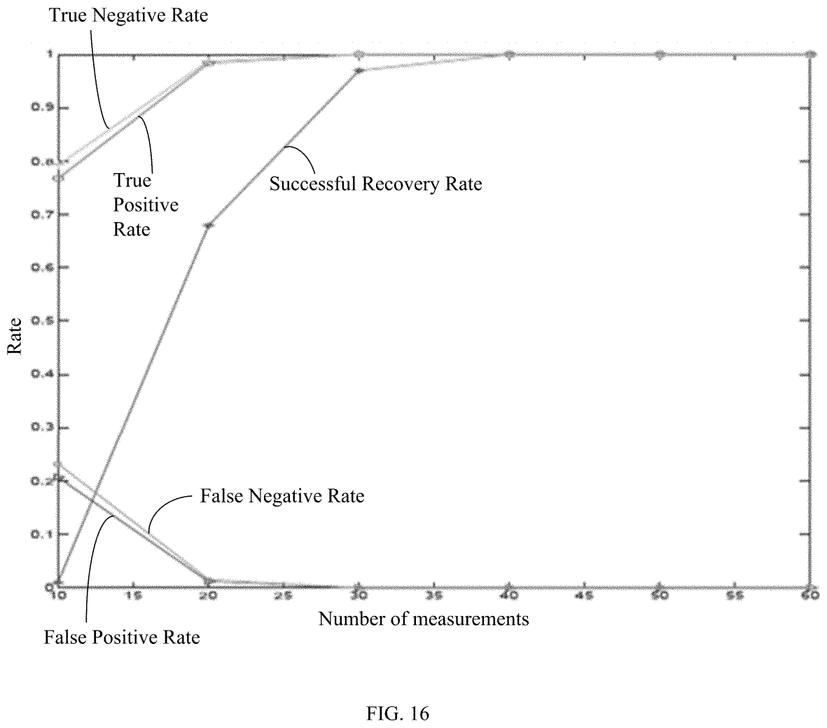

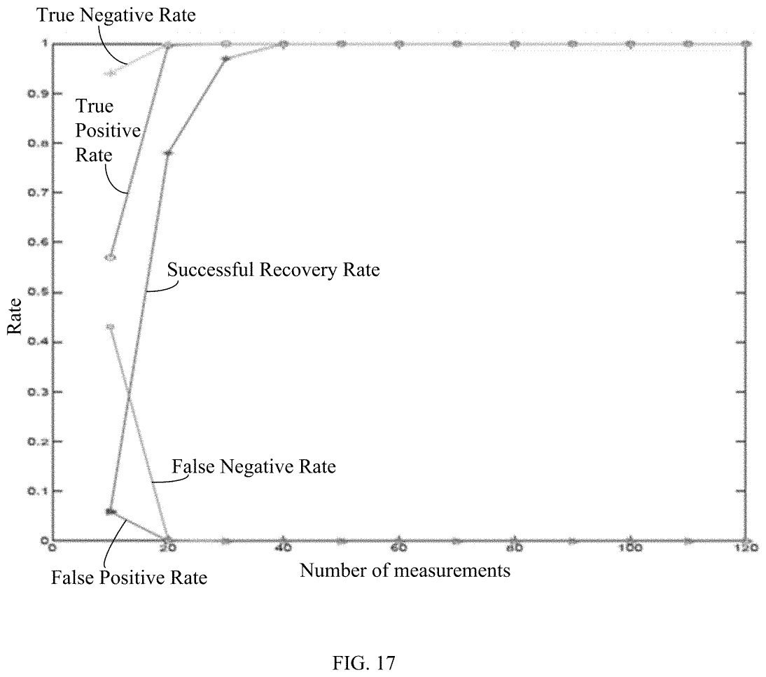

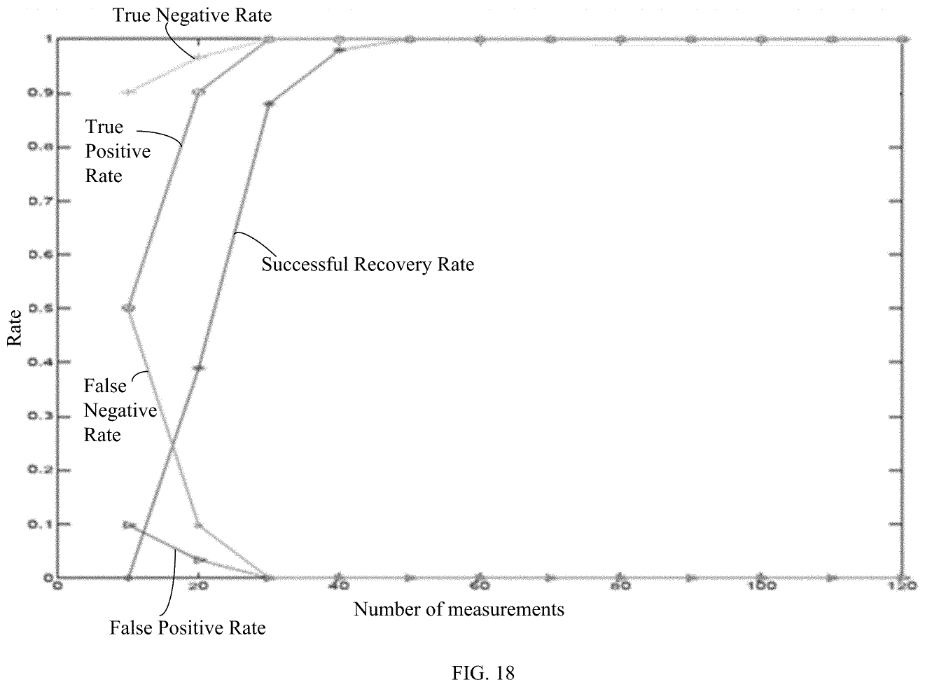

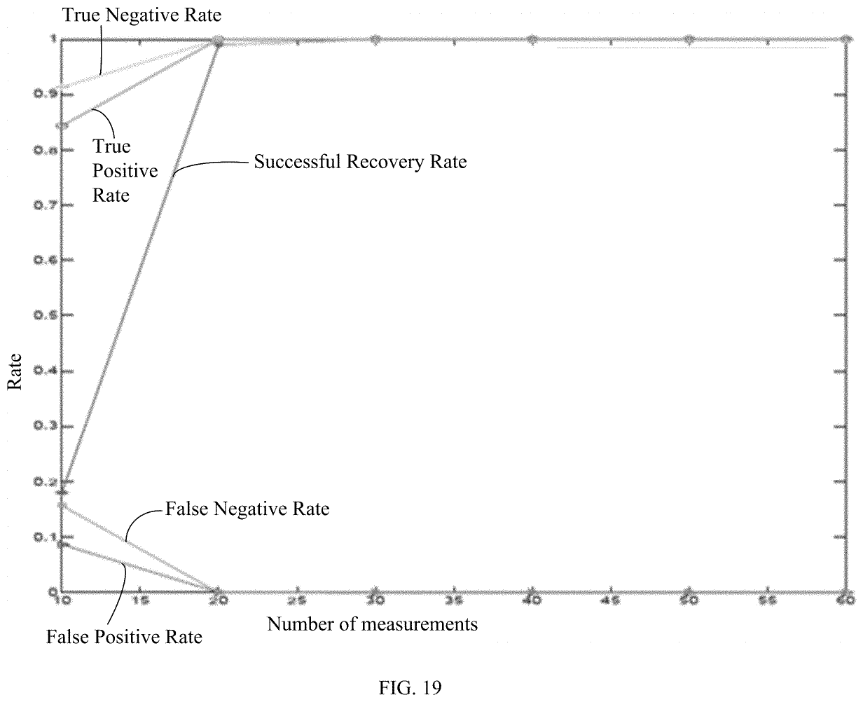

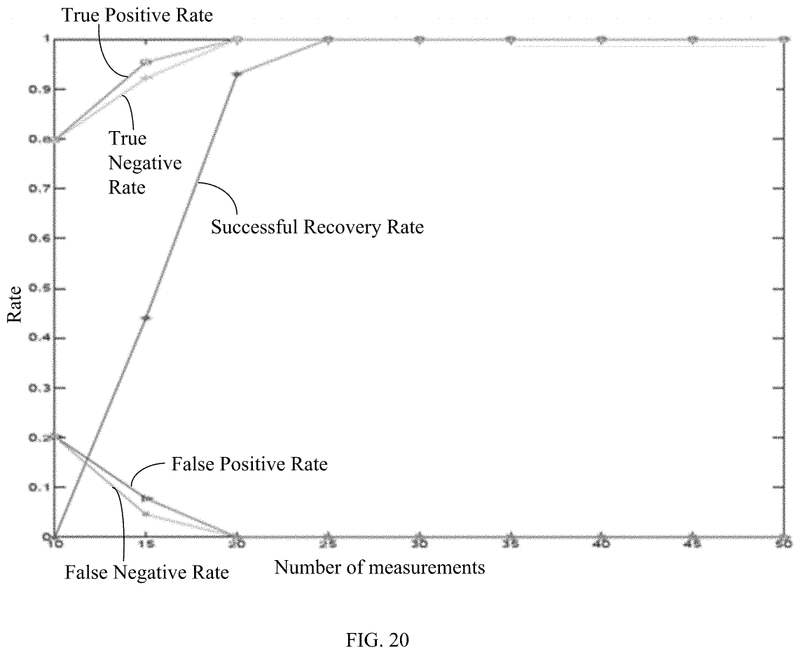

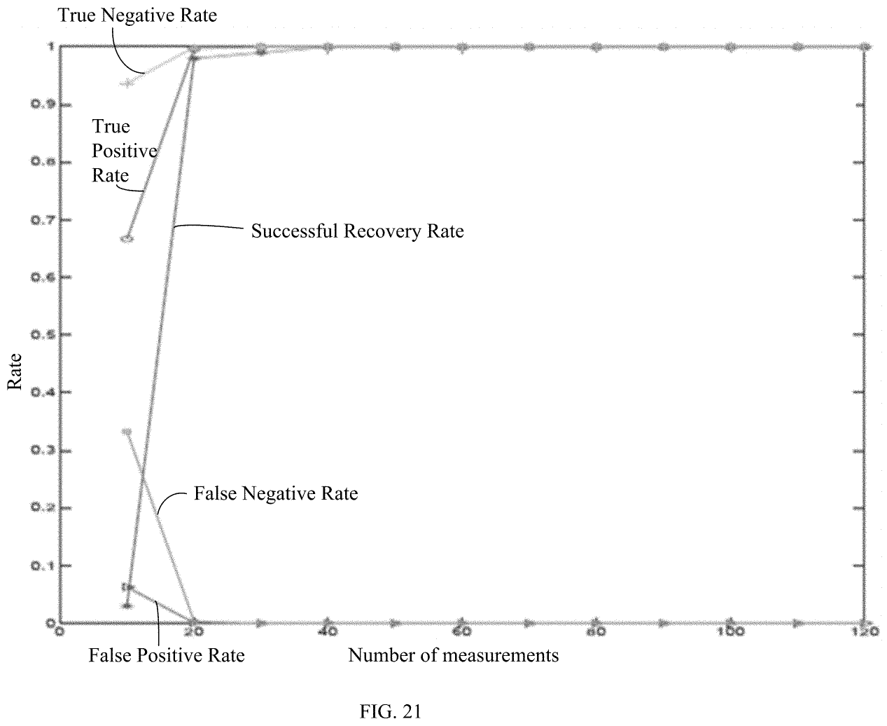

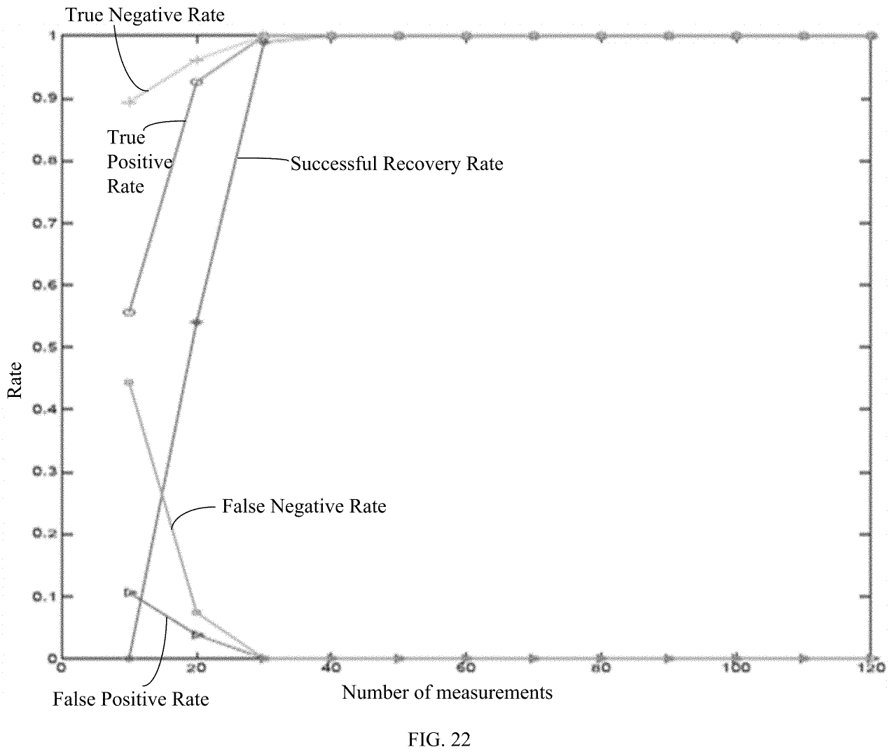

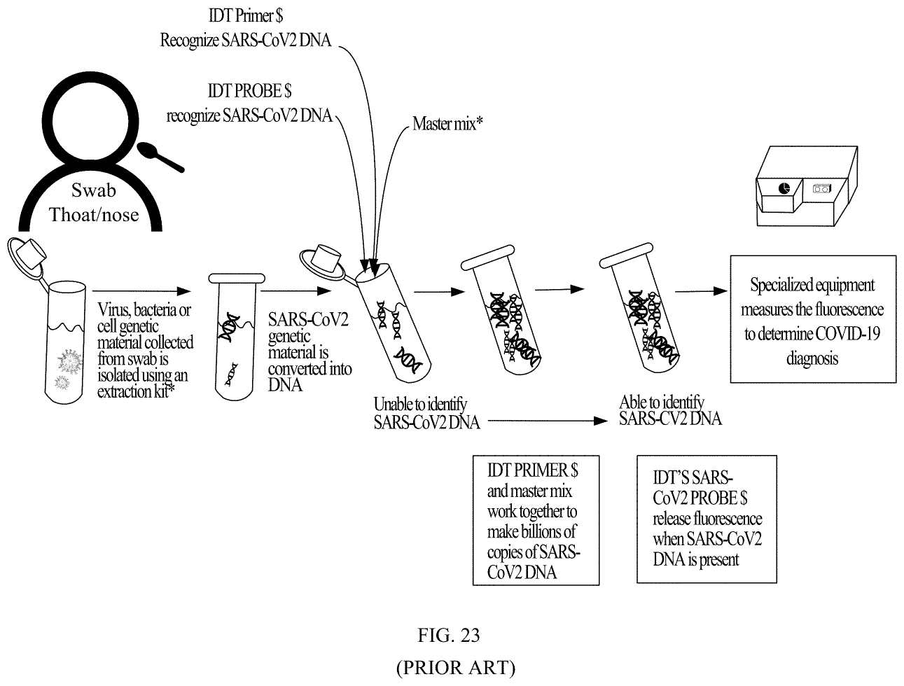

We also conduct experiments with noisy measurements, and the signal is recovered from noisy measurements by solving

$${min}_{x ∈ {n}^{}} {‖ x ‖}_{1} , \\ s . t . {‖ Ax - y ‖}_{2} \leq ϵ , x \geq 0 , \qquad ( 7 )$$

where ϵ>0 is a parameter tuned to noise magnitude, and y∈ [symbol] ^{m} is the noisy measurement vector. We follow the same setup as in previous section expect that for each trial of each set of parameters (m; n), we add randomly generated noise vector v with normalized magnitude 10⁻³ to the measurements, namely y=Ax+v. For each trial of each set of parameters, we treat the recovery as successful if it achieves a reconstruction error less than 10⁻². The results of the recovery probabilities, false positive rates, and false negative rates are shown in the following figures from FIG. 15 to FIG. 22. FIG. 12 shows the results for k=2 and n=200, demonstrating a possible increase of throughput by 10 times.

We can see that similar increases in testing throughput are also observed as in the noiseless cases. In fact, for a large range of reasonable noise levels, we can observe similar increases in testing throughput with low false positive rates and false negative rates.

In another experiment, we numerically evaluate the performance of exhaustive search in detecting viruses. We take n=40 and k=2, and the number of measurements is taken as m=5, 6, 7, 8, 9, and 10. For each set of (m; n; k), we run 10 trials. In each trial, the pooling matrix is a Bernoulli random matrix. The measurement result is contaminated with random noise normalized to have a magnitude of 10⁻³. A trial is considered to have successful recovery if the recovery error is less than 10⁻² in the noisy case. In exhaustive search, since the true signal has sparsity of k, we will simply perform brute force calculations over all the possible sets of k infected persons. For each possible such set of cardinality k, we extract the corresponding columns from the measurement matrix. By doing this, we get an overdetermined system, and solve it via the least squares method. There are totally

$$( \begin{matrix}n \\ k\end{matrix} )$$

possible such sets, which means we need to solve the least square

$$( \begin{matrix}n \\ k\end{matrix} )$$

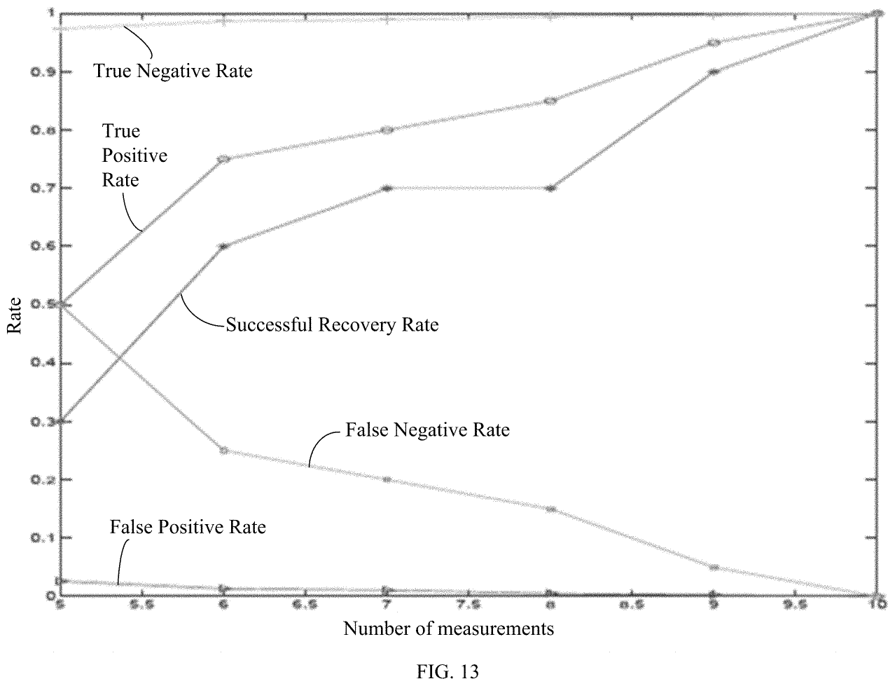

times for each trial. The results are shown in FIG. 13. As we can see, using only 10 measurements, the false positive rate and false negative rates are very low (in fact 0 in this experiment). That amounts to a factor of

$$\frac{40}{10} = 4$$

speedup in throughput of the test.

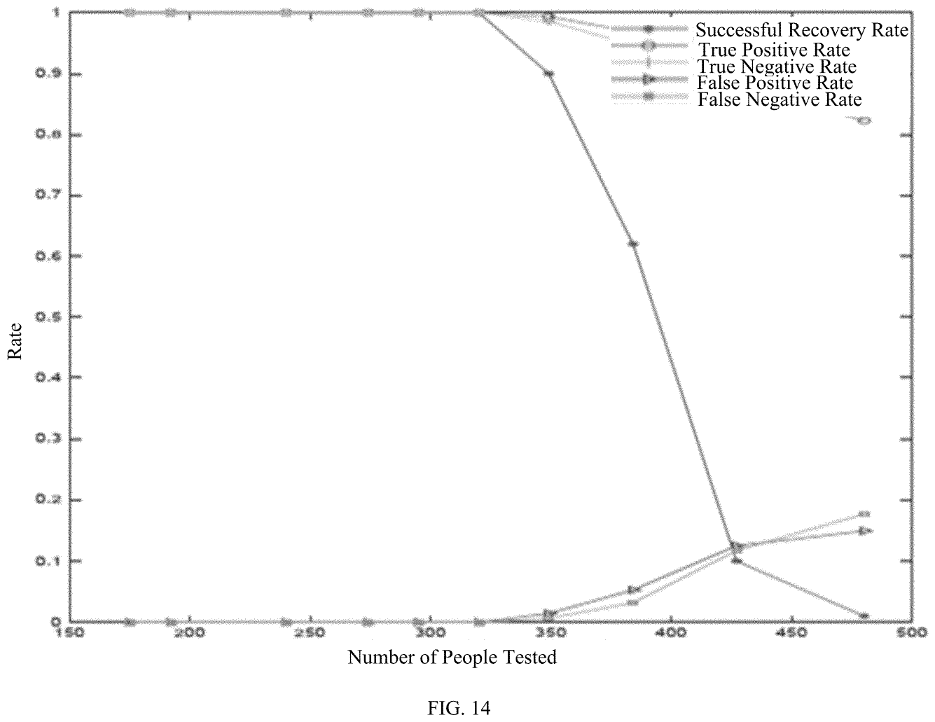

We now look at the testing data of COVID-19 viruses from the state of Iowa. The rate of testing positive is around 8.7 percent by early April, meaning among all the tests carried out, 8.7 percent of them came back with a ‘positive’ result. We consider a microplate of 96 wells and assume that the PCR machine can analyze 96 samples in one operational period. Then we do a computational experiment to answer, “using compressed sensing, for how many people these 96 compressed sensing (pooling) samples can correctly identify all the carriers of viruses present in that group of people?” In this experiment, we x the number of measurements, namely m, as 96. Then we vary the number of people n, and randomly pick 8.7 percent of them (namely k=ceil(0.087 n), where cell(⋅) is the ceiling function) as virus carriers. We accordingly generate the virus quantity vector x. We plot the successful recovery rate of x, the false positive rate and false negative rate as functions of n in FIG. 14. As n increases, there are more virus carriers, and false positive rates and false negative rates are expected to increase when m=96 is fixed. We observe that for n≤300, these false positive rates and false negative rates stay very low. This means that, when 8.7 percent of people have viruses, using compressed sensing, the throughput of testing can grow to as much as

$$\frac{300}{96} \approx 3$$

times. For both Bernoulli random matrices and expander graph based matrices with 7 ‘1’s in one column, we observe similar behaviors.

When the percent of people carrying viruses decreases, say to 1 percent, compressed sensing can even increase the throughput by more than 10 times.

### 4. Discussions

Here the focus has been on non-adaptive compressed sensing, which can have the advantage of minimizing the latency in obtaining the test results for tested persons. However, it is totally possible to increase the throughput of testing by using adaptive measurements for compressed sensing, as adopted in [28][38] for group testing.

### Part 3: Error Correction Codes for Increasing Reliability of COVID-19 Virus and Antibody Testing Through Pooled Testing

Here, we consider a novel method to increase the reliability and capacity of Covid-19 virus or antibody tests by using specially designed pooled sampling methods. Specifically, instead of testing nasal swabs or blood samples from individual persons, we propose to test a number of mixtures of samples from many individuals. This potentially allows us to (a) determine the infection status for many individuals using significantly fewer tests than individuals, and (b) correct for some fraction of incorrect test results. The idea is to take advantage of (a) the likely low rate of infection in the population i.e. the likelihood that only a small fraction of the tested population is actually infected at any time, and (b) the statistical independence of incorrect results in multiple tests. We use ideas from the theories of compressed sensing and error correction coding to design efficient sample mixtures to minimize the number of tests needed, and to correct for some proportion of incorrect test results. Our approach also allows a trade-off between the diagnostic accuracy and testing capacity i.e. we can in theory make the diagnostic accuracy arbitrarily high by increasing the number of tests. Simulations demonstrate the effectiveness of the proposed method in simultaneously achieving substantial increases in testing capacity and diagnostic accuracy.

### I. Introduction

In the absence of a vaccine to the Covid-19 coronavirus, the experience of public health authorities in several countries has shown that large-scale shutdowns can only be safely ended if a systematic “test and trace” program [32][43] is put in place to control the spread of the virus. This, in turn, is predicated on the widespread availability of mass diagnostic testing. However, most countries including the US are currently experiencing a. scarcity [34] of various medical resources including tests [25].

### A. Background: Covid-19 Virus and Antibody Tests

The most common tests for the Covid-19 virus currently used in the US and recommended by the CDC are swab tests. These tests use the Reverse Transcription Polymerase Chain Reaction (RT-PCR) process to selectively amplify DNA strands produced by viral RNA specific to the Covid-19 virus. The RT-qPCR process which is considered the gold standard for the detection of mRNA consists of three distinct steps:

(1) reverse transcription of RNA into cDNA, (2) selective amplification of a target DNA fragment using the Polymerase Chain Reaction (PCR), and (3) detection of the amplification product. While the simple “end-point” version of PCR only allows binary detection (presence or absence) of a target RNA sequence, the real-time or quantitative version of the PCR process (qPCR) [26] also allows the quantification of the RNA i.e. it produces an estimate of the quantity of the RNA material present in the sample [44].

Some researchers [43] have proposed the Reverse Transcription Loop-Mediated Isothermal Amplification (RT-LAMP) as a potentially cheaper and faster alternative to RT-PCR for swab tests. While we focus on tests based on the RT-qPCR process, the methods proposed herein are also compatible with RT-LAMP [36] and other DNA amplification methods.

The PCR-based virus tests are highly sensitive (i.e. have low rates of false negatives) as well as specific (i.e. successfully differentiates between the Covid-19 virus and other pathogens and therefore shows low false positive rates). However, pooled sampling methods require sample dilution and additional preparation that may potentially result in degraded sensitivity as well as specificity.

In addition to tests for an active Covid-19 viral infection, there has also been interest in testing for the presence of antibodies to the Covid-19 virus. These antibody tests can show that a person had some time in the past been infected with the Covid-19 virus and may have some immunity to the virus. Virus and antibody tests complement each other nicely: virus tests allow us to determine if an individual needs to be quarantined, whereas antibody tests may tell us when an individual is not at risk of getting infected.

Antibody tests typically use blood samples (unlike virus tests that use nasal swabs), and typically use an enzyme immunoassay process such as ELISA (enzyme-linked immunosorbent assay) [45]. ELISA's tests typically show high sensitivity; however, some of the early antibody tests that were commercially introduced for Covid-19 may have issues with selectivity [45].

### B. Increasing Testing Capacity

One simple method to increase the effective testing capacity by testing pooled samples of a number of test subjects collectively instead of testing samples from each person individually. In the simple version of this idea called “group testing” [16], a single negative test result on a pooled sample immediately shows that all individuals in that pool are infection-free. Thus, individual tests only need to be performed when a specific pooled test sample yields a positive test result. When the rate of infection in the population is low, this method allows us to reduce the total number of tests per subject so the throughput of the existing testing infrastructure is increased [27]. Pooling tests have been successfully used for diagnostic testing for infectious diseases in the past [18] [17].

The current testing bottlenecks in the Covid-19 crisis has led to a resurgence of interest in using group testing methods for Covid-19 diagnosis. Specifically, there have been recent studies [40][38][46][42] into adapting pooling methods similar to [16] for Covid-19 testing. Preliminary studies on the Covid-19 virus also show that pooling samples [41] can be effective with existing RT-PCR tests.

In our own recent work [47], we proposed a different approach based on the compressed sensing theory [23][1][2] for detection of viruses and antibodies using pooled sample testing. Our compressed sensing method is more powerful and can achieve higher efficiencies and better performance than group testing. Indeed, group testing is a simple special case of the more general compressed sensing method.

The basic idea behind the compressed sensing pooled sampling method is to prepare a set of mixtures of several individuals' swab specimens, where the mixtures are carefully chosen to be different from each other in such a way that, under the assumption that only a small fraction of the individual samples have non-zero viral RNA, each individual's diagnostic status can be determined by testing a number of mixtures much smaller than the number of individuals.

### C. Increasing Testing Accuracy

Our simulations in [47] show that the compressed sensing method is effective in achieving a significant increase in testing capacity. We take this idea further and show that the compressed sensing method can also increase the accuracy of diagnostic tests by taking advantage of redundancy in the pooled sample test results to correct for some number of incorrect test results.

To motivate this idea, consider a population of N individuals. Let bᵢ∈{0, 1}, i=1 . . . N represent the infection status of the i-th individual in the population i.e. bi=1 indicates individual i is infected with the virus. The information vector b=[b₁, b₂, . . . , b_{N}]∈{0,1}^{N} represents the infection status of the population as a whole.

Let p denote the infection rate in the population:

$$p = E ( \frac{1}{n} {\sum }_{i = 1}^{N} {b}_{i} ) .$$

While the information vector h can be represented by the N information bits bᵢ, i=1 . . . N, an elementary result from information theory shows that the entropy of the information vector is much smaller than N bits, when the infection rate is low:

h(b)≡−Np log₂ (p)−N(1−p) log₂ (1−p)<<N; if p<<1 (1)

where we assumed that each individual in the population independently has a probability p of being infected. The entropy h(b) represents the number of bits required to losslessly represent the information in b.

Thus, (1) can be interpreted as a theoretical justification for pooled sample testing: in theory, we only need tests that deliver a total of Nₜ=h (b) bits of information in order to fully recover the infection status bᵢ of every individual in the pool. If the tests are binary i.e. only indicate positive/negative infection status and are completely error-free, then in theory we can fully diagnose all N individuals with as few as h(b) such tests.

If the test provides richer non-binary results (e.g. quantification of viral RNA concentration from RT-qPCR tests), in theory the number of tests needed may be much smaller than h(b).

In this sense, pooled sample testing methods such as our compressed sensing method, can be thought of as data compression codes. However, the tools of information theory allow us to design codes that have much more powerful capabilities than just lossless data compression. In particular, we can generalize from lossless data compression to codes that can perform data compression combined with error correction. In the context of virus testing, this means a class of pooled sample testing techniques that can achieve accurate diagnostic results even with tests that are individually highly error prone.

We show herein a class of compressed sensing pooled sample testing methods that do exactly this: increase testing capacity (data compression) combined with increased diagnostic accuracy (error correction). In other words, we demonstrate a method of pooled sample testing that requires fewer tests in aggregate, yet delivers more accurate diagnostic results than separately testing each individual.

### II. Problem Statement

In this section, we will give a mathematical formulation of performing robust virus testing through error correction code. We will focus on describing the idea of error correction code for virus testing through quantitative pooled testing, even though the idea of error correction code can be extended to traditional qualitative pooled testing.

The quantitative modeling of the pooled testing problem requires the application of real-time polymerase chain reaction (real-time PCR) which is built on top of the PCR and conducted in a thermal cycler. The real-time PCR can give quantitative measurements of the amplified DNA copies by using fluorescent reporter in each PCR cycle during which the DNA template can be doubled, and the strength of the signal from fluorescent reporter is proportional to the number of amplified DNA molecules. A threshold of 35 times the standard deviation of the background noise is used for detecting the existence of virus, and the number of cycles which achieves a value no less than the threshold is called the threshold cycle Cₜ.

Assume we get totally n samples for n subjects with one sample for each, and we will perform m<<n tests to determine the existence of COVID-19 viruses in these samples. We denote by x∈[0, ∞)ⁿ the quantitative measurement of the DNA sequence if we use the real-time PCR after initial several cycles. In each of the in tests, we will obtain a combined sample by mixing the samples from multiple testees. We use a matrix P∈{0, 1}^{m×n} to denote the participation of n samples in m tests, i.e. the sample of the j-th testee participates in the i-th test if Pᵢⱼ=1, and it will not be used in the i-th test if Pᵢⱼ=0. This means that the number of 1's in the j-th column of P is the number of tests that the sample of j-th testee will participate, and this further requires an allocation scheme for a testee's sample, We will model the allocation of the testee samples by W∈[0, 1]^{m×n}, and each Wᵢⱼ is the portion of the j-th sample used in the i-th test. With those setups above, we get a measurement matrix as

A=P⊙W, (2)

where ⊙ represents Hadamard multiplication.

The corresponding mixed samples A×∈[0,∞)^{m} will then be used for m tests after going through the real-time PCR process to get enough copies of the DNA sequences. Due to the potential background noise and gross errors such as operational mistakes in the test laboratories, the final quantitative measurements y∈ [symbol] ^{m} from the real-time PCR

y=ƒ(Ax)+v+e, (3)

where ƒ(⋅): [symbol] ^{m}→ [symbol] ^{m}, v∈ [symbol] ^{m}, and e∈ [symbol] ^{m} characterize the copying process, the background noise, and gross errors. For example, if we assume that in each test, the amplification folds are the same for all the testees' samples which participate in the test, then the y can be formulated as

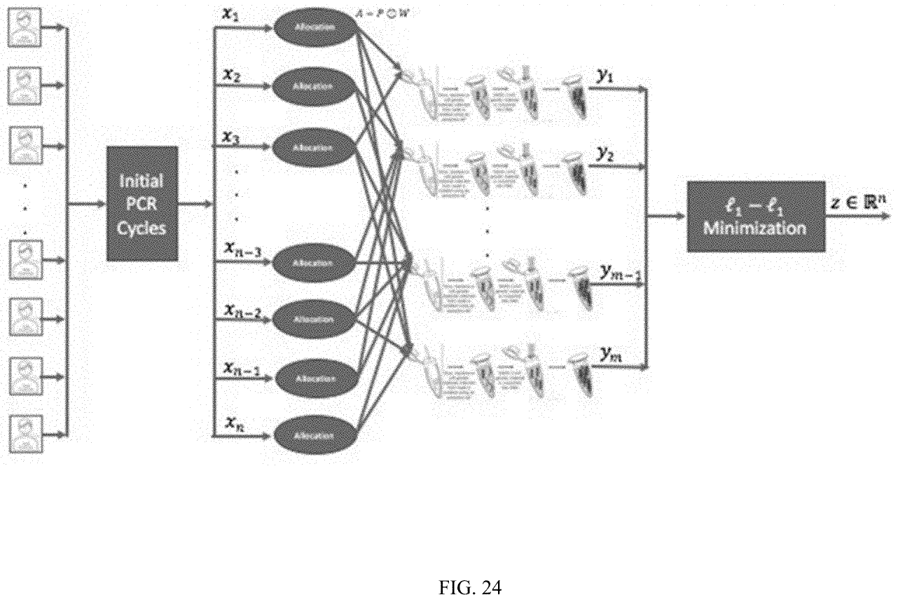

y=GAx+e+v, (4)

where G is a diagonal matrix determined by the number of cycles performed for amplification. See FIG. 24 for the relation between the quantitative measurement and the number of cycles.

Our goal is to recovery the sample measurements x∈[0,∞)ⁿ for n testee from in tests measurements y∈ [symbol] ^{m}. Once the x∈[0,∞)ⁿ is recovered, the amplified measurements for the n testees will be

xₐₘₚ=Gx (5).

A threshold τ of 35 times the standard deviation of the background signal noise can then be used for xₐₘₚ to determine whether a testee is infected. For example, if (xₐₘₚ)ᵢ≥τ, then we can claim the i-th testee is infected.

We now make some extra assumptions which are commonly used in practice. According to [47][48], a measurement matrix from the expander bipartite graph can achieve good practical performance with well-sound theoretical justifications, and we will specify the matrix P as such matrices, i.e., a sparse binary matrix. The sparsity of matrix P is characterized by the number of 1's in each column which is determined by taking practical considerations such as there should not be too many 1's since we do not want a testee to get involved in too many tests. There should also be enough 1's in each column so that we can get enough information about a testee. In the extreme case where a testee participates in none of the test, we cannot make any conclusions about whether the testee is infected or not. Due to the above constraints, we will design the matrix P based on the ideas in [4][5]. Though we have freedom to design the allocation matrix W, we will use an even-allocation scheme to get such a matrix. Thus, if the j-th testee is involved in c tests, then the j-th column of P has only c 1's, and the j-th column of P will have nonzero values at the corresponding location being

$$\frac{1}{c} .$$

The low infection rate among population in practice allows us to assume that the sample measurement x∈[0,∞)ⁿ is sparse or approximately sparse, i.e., most of its entries are zero (or extremely close to zero). The scarcity of making mistakes by the laboratory professionals implies that the gross error v∈ [symbol] ^{m} is also sparse, and we will further assume the background noise has a very low-level power or energy.

Under all these assumptions, we can formulate the problem of recovering x∈ [symbol] ⁿ from y∈ [symbol] ^{m} with m<n as

minimize ∥z∥₀+λ∥y−GAz−u∥₀

subject to ∥u∥₂≤ϵ,

z≥0, (6)

where ∥z∥₀ is the number of nonzero elements in z, λ→ [symbol] is a tuning parameter for controlling the tradeoff between ∥z∥₀ and ∥GAz−y−u∥₀, the ∥u∥₂ is the [symbol] norm of u, ξ≥0 is the tolerance for noise, and the x 2:′.0 means that every element of x is nonnegative. In (6), we used z as an estimate for x and u as an estimate for v, and y−GAz−a is an estimate for e.

Due to combinatorial characteristic of ∥⋅∥₀, solving (6) is in general NP-hard, and the ∥⋅∥₁ can be used as a relaxation technique in practice to achieve good performance without much computational difficulties [19][2]. Thus, we can reformulate (6) as

Minimize ∥z∥+λ∥y−GAz−u∥₁,

subject to ∥u∥₂≤ξ,z≤0 (7)

where ∥z∥₁ is the sum of the absolute value of all the elements in z, and we will refer (7) as [symbol] - [symbol] minimization. Once the estimate for is obtained, we can get an estimate of xₐₘₚ via (5), i.e.,

zₐₘₚ=G_{z} (8)

If (zₐₘₚ)ᵢ≥τ where τ is the threshold value, then we claim the i-th testee is infected and positive. Otherwise, we declare negative result for the testee.

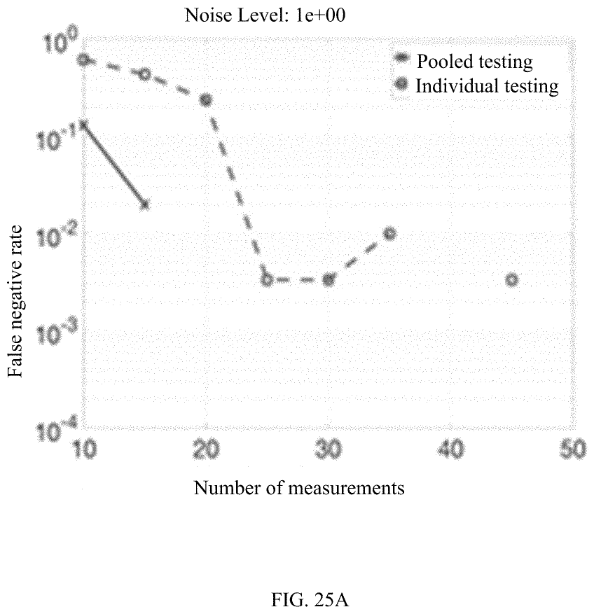

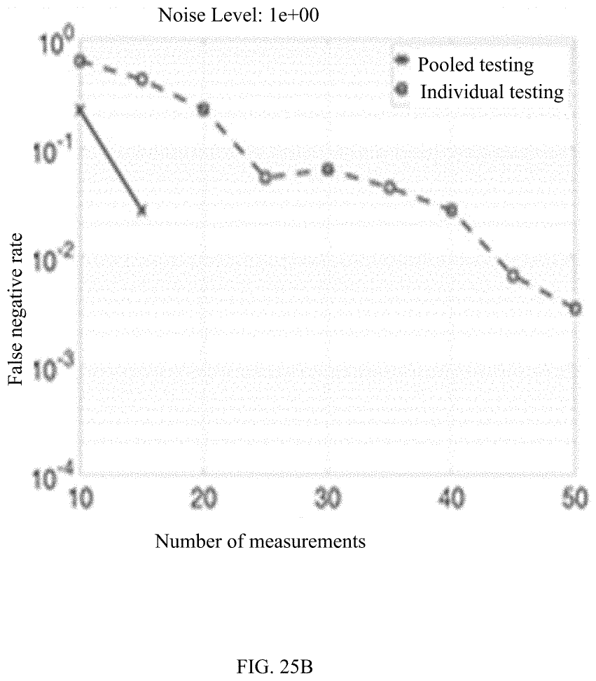

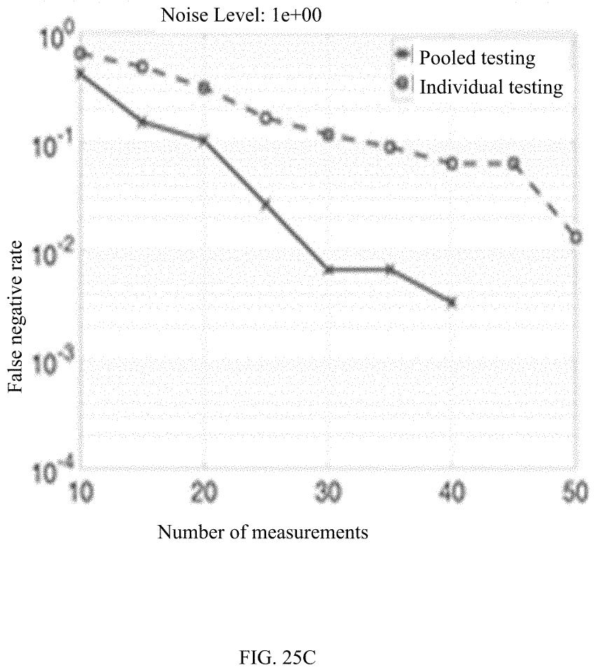

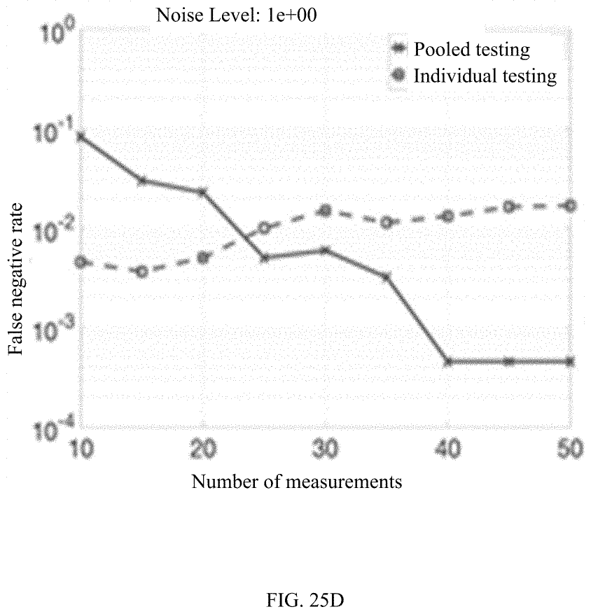

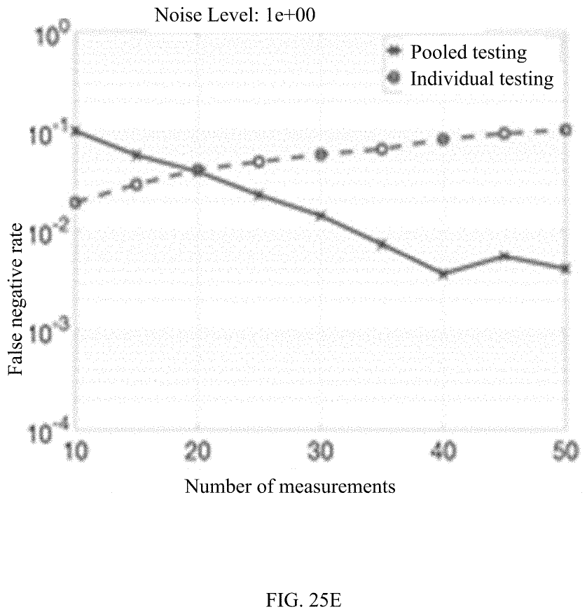

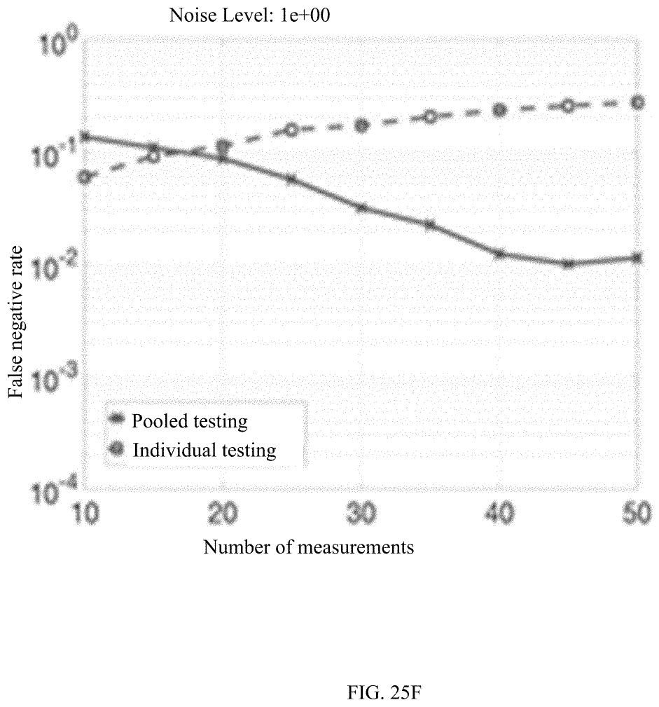

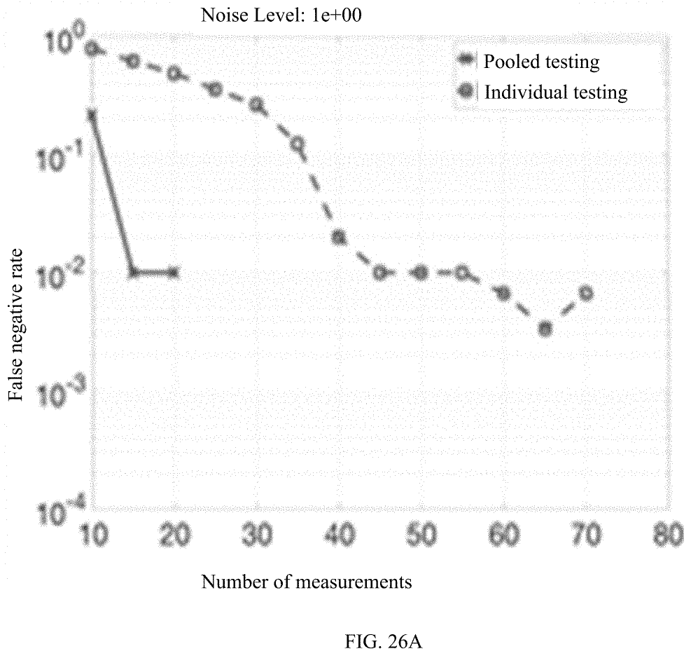

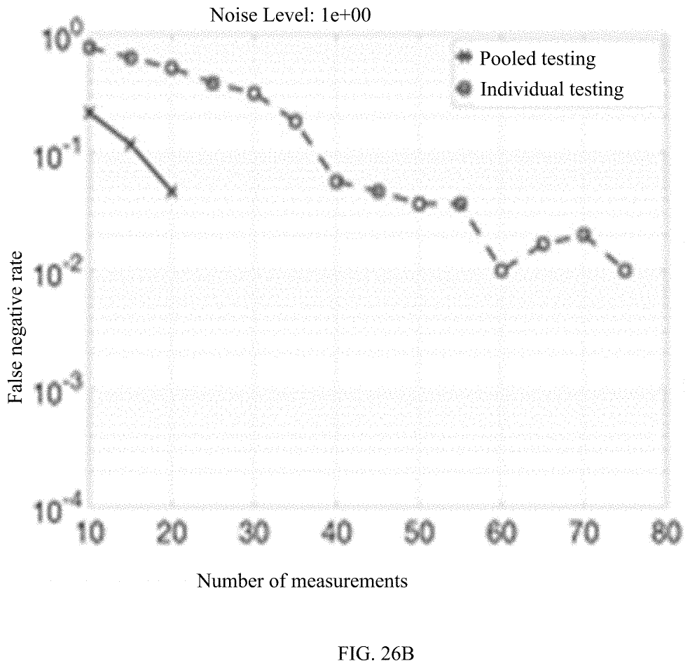

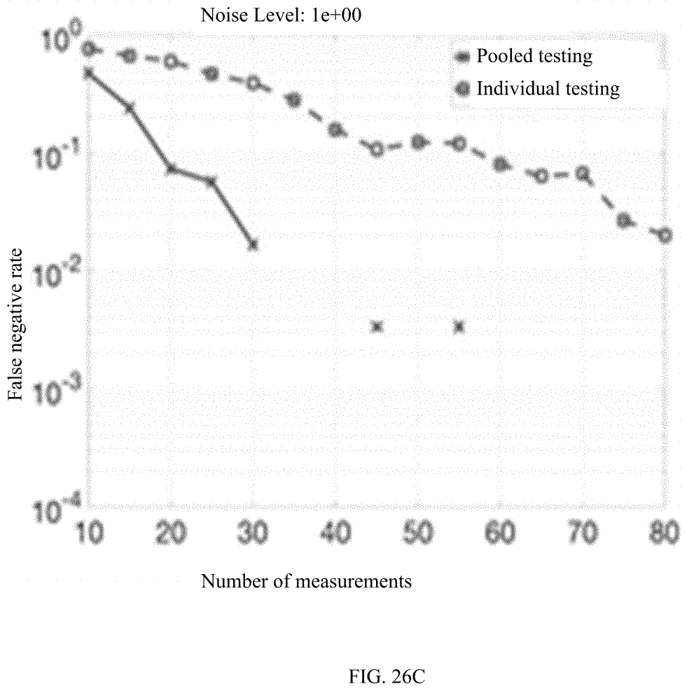

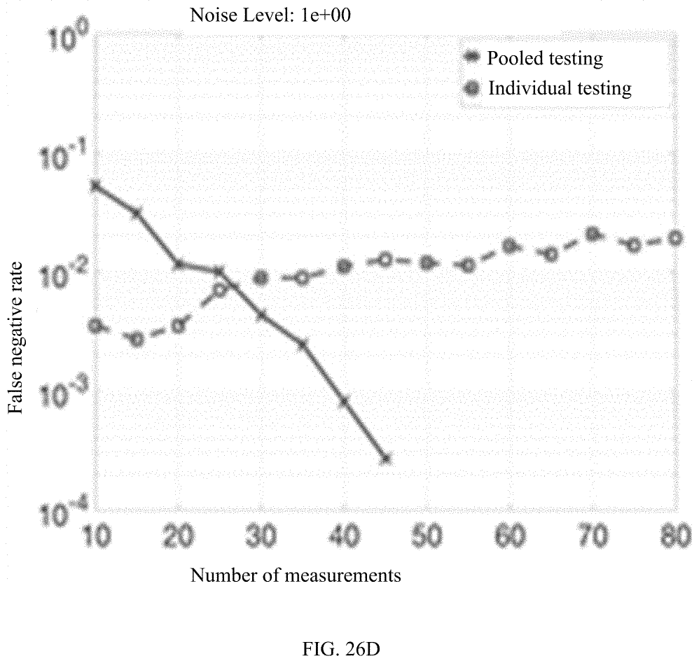

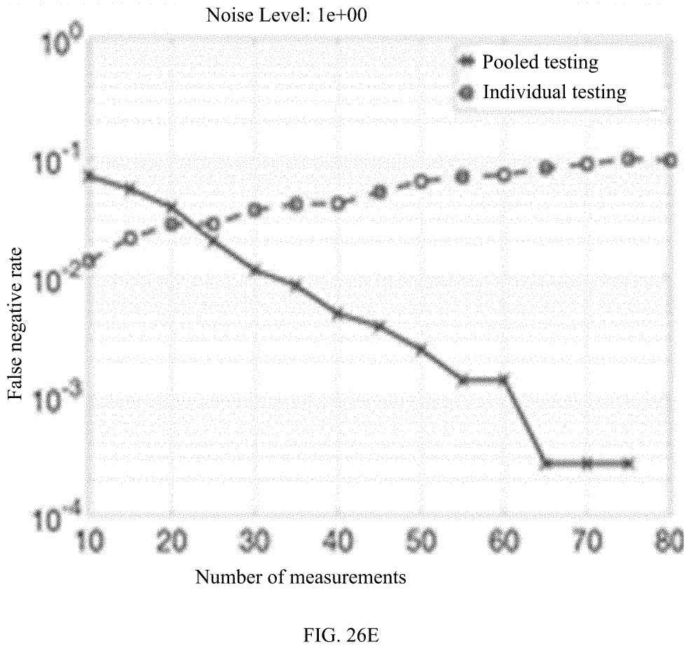

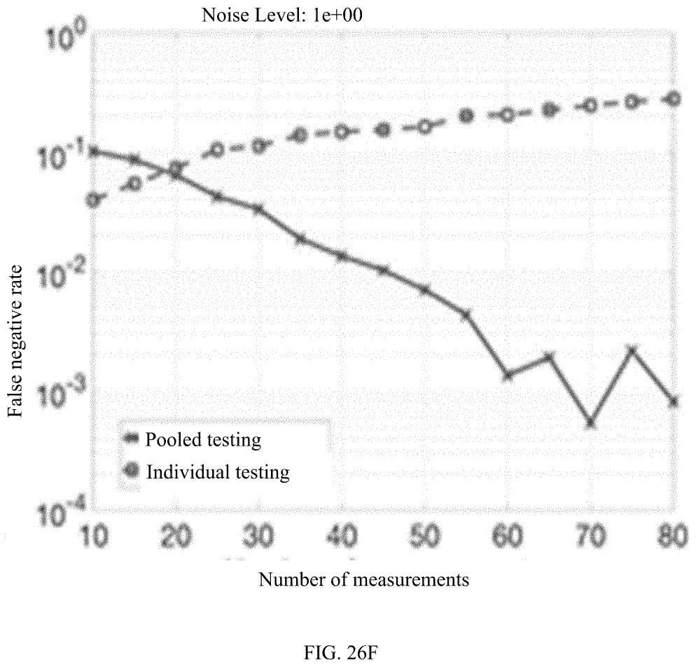

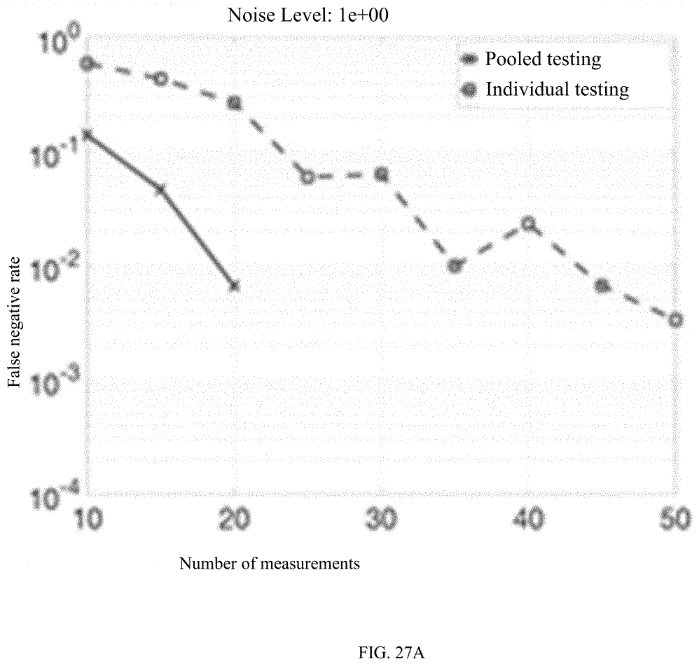

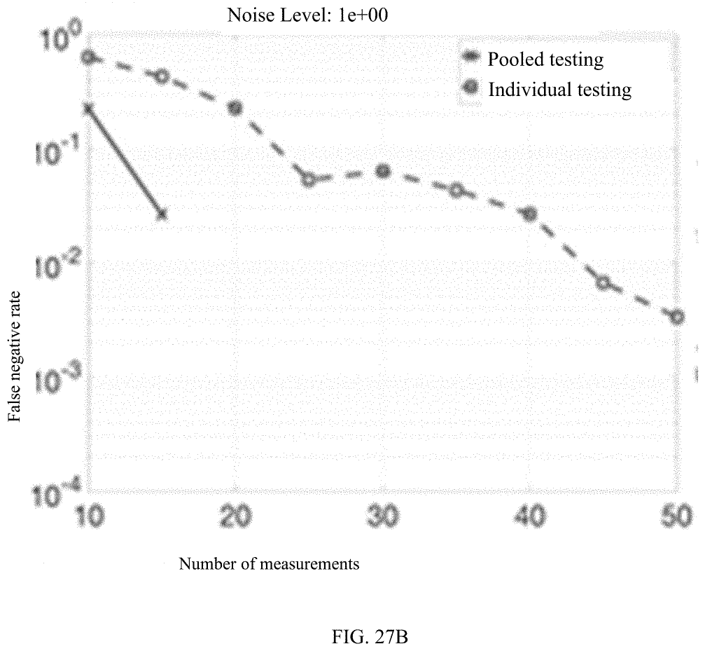

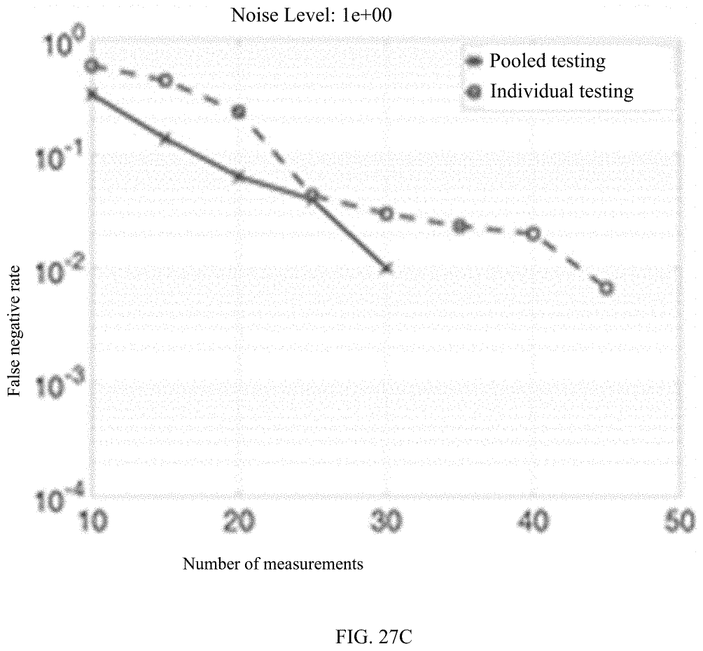

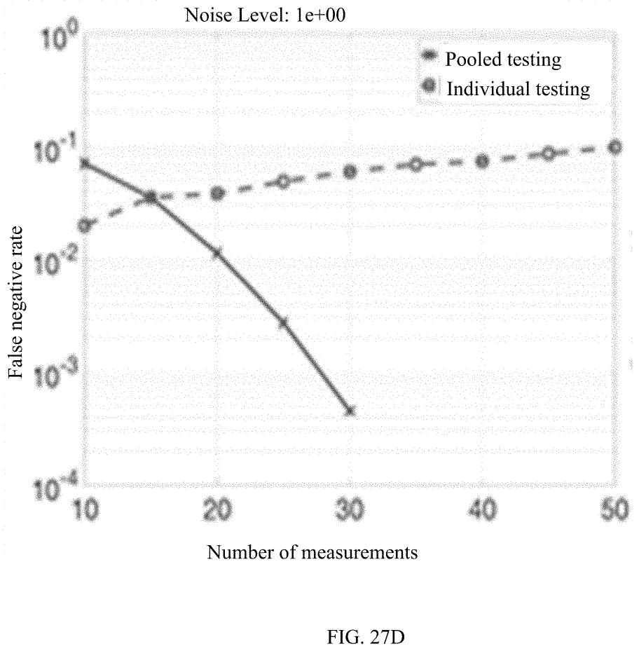

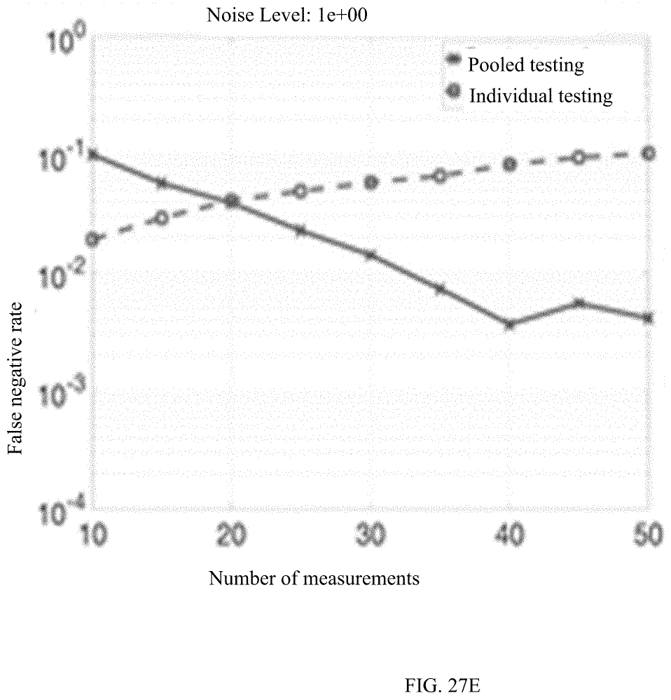

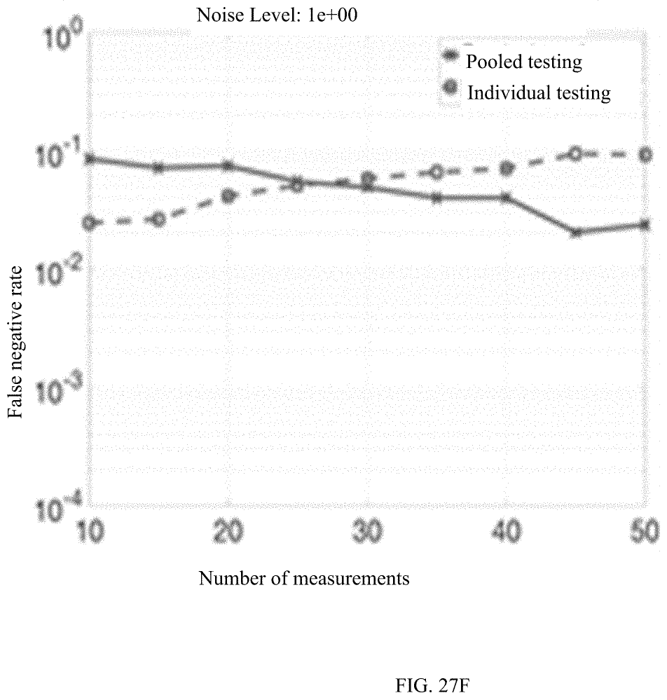

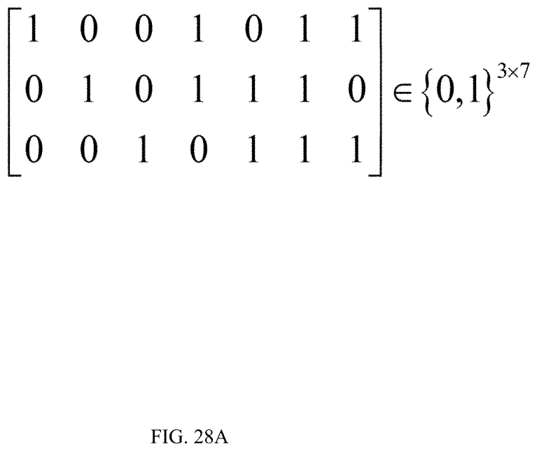

There is a large volume of literature which proposed ideas for solving (7) under certain conditions such as the restricted isometry property and the null space condition. These ideas range from using off-the-shelf softwares such as CNA [49], to algorithms specifically designed for [symbol] - [symbol] minimization such as the homotopy method and iteratively reweighted least square algorithm [48]. We will use the CNA [49]. The overall framework of the proposed testing approach is illustrated in FIG. 25.

### III. Numerical Experiments

In this section, we conduct numerical experiments in order to evaluate the performance of our proposed method, which is the Covid-19 pooled testing introduced in (7). In order to reflect pooling operation, we randomly choose Bernoulli matrices having 1 with the probability 0.5. We assume that the DNA amplification is processed evenly for all tests; thus we treat the matrix G in (7) as the identity matrix. The numbers of people tested are set to 25 and 40, i.e., n=25 and 40. We consider a scenario where k out of n people have Covid-19 virus by setting randomly chosen k elements in x∈ [symbol] ⁿ to be positive and other n−k elements to zero. The value of the non-zero elements is chosen within [5, 10] uniformly at random. We consider the sparsity level k from 1 and 6 in the simulations. For the outlier error, denoted by e in (4), we take into account three probabilities of the outlier error, denoted by Pₒᵤₜ, to be 1%, 5%, and 15%. Hence, the vector e in (7) has non-zero elements with the probability of Pₒᵤₜ. The support and the value of the non-zero elements in the outlier error are also chosen uniformly at random following N(2, 5). The Gaussian noise vector v in (4) is set to following N (0, σ²), where the noise level a 2 is varied from 5e-1 to 2e0. Even with the Guassian noises and the outlier errors, we make sure that the measurement y in (4), which represents the number of DNA of Covid-19 virus, to be positive by changing the sign of the error or the noise, if necessary.

For comparison, we generate an individual testing model for the i-th testee as follows:

yᵢ=x_{mod(i,n)}+eᵢ+vᵢ,i=1,2, . . . ,m (9)

where yᵢ is measurement, xᵢ is the number of DNA related to Covid-19, eᵢ is an outlier error, and vᵢ Gaussian noise following N(0, σ²), where the noise level σ² is also varied from 5e-1 to 2e0. Since we deal with small number of measurements, if m<n, namely, there is someone who doesn't receive the PCR test, then, we consider the person as Covid-19 negative.

Additionally, if we have two testing results for one testee and at least one result is identified as being positive, we consider the testee as Covid-19 positive. This is because of not to miss the Covid-19 positive cases by doing the testing conservatively. The number of measurements, denoted by m, is varied from 10 to 50 in n=25 and from 10 to 80 in n=40. Thus, in our individual testing scenario, the maximum number of tests for a testee is two.

For both the pooled testing and the individual testing, we run 100 random trials for each measurement and record the False Negative Rate (FNR) and the False Positive Rate (FPR), which are computed in average out of 100 trials as follows:

$$F N R = \frac{Number of negative cases in people with Covid \text{-} 19 virus}{Number of people having Covid \text{-} 19 virus} \\ F N R = \frac{Number of negative cases in people without Covid \text{-} 19 virus}{Number of people not having Covid - 19 virus}$$

Hence, the FNR represents the rate of cases where people having Covid-19 virus are identified as Covid-19 negative, which can be a critical error in Covid-19 testing. For FPR, it is interpreted as the rate of cases where people not having Covid-19 virus are identified as Covid-19 positive due to noise or error in testing procedure. The FPR can be an important indicator in Covid-19 antibody testing. Hence, the lower both FPR and FNR represent the better testing performance in detecting virus and checking antibody. Additionally, if one method achieves the same FNR and FPR with a smaller number of measurements than the other, then, the method will be better than the other. This is because the number of measurements is related to the throughput of testing, and the high throughput testing allows us to increase the capacity of the number of tests in a limited time. Therefore, through the various simulations, we will compare the FNR and the FPR of the pooled testing against those of the individual testing as the number of measurements increases in different noise levels and outlier error rates.

### A. Different Probability of Outlier Errors

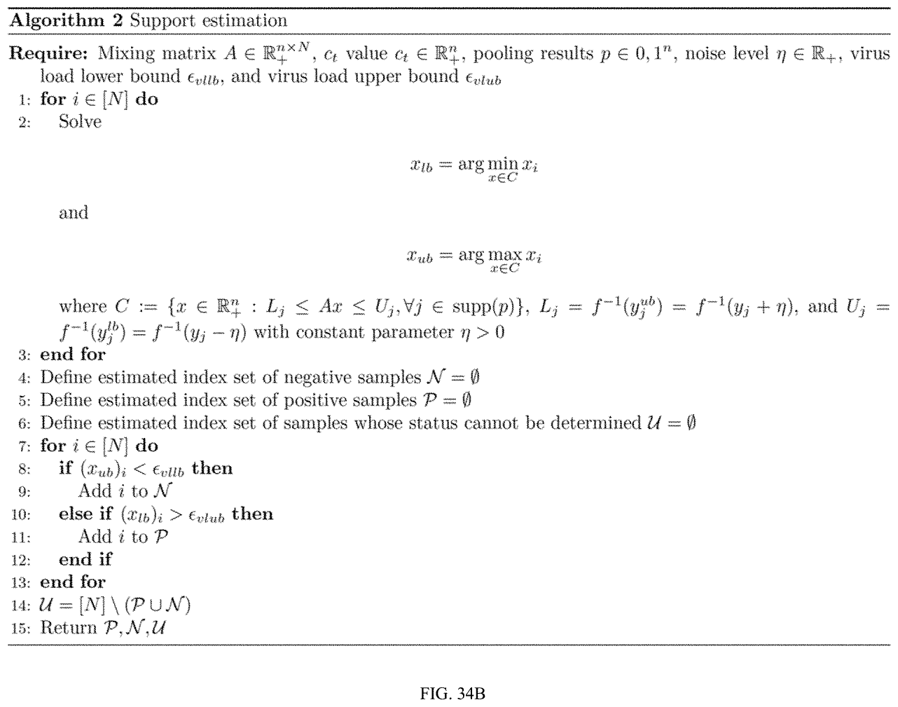

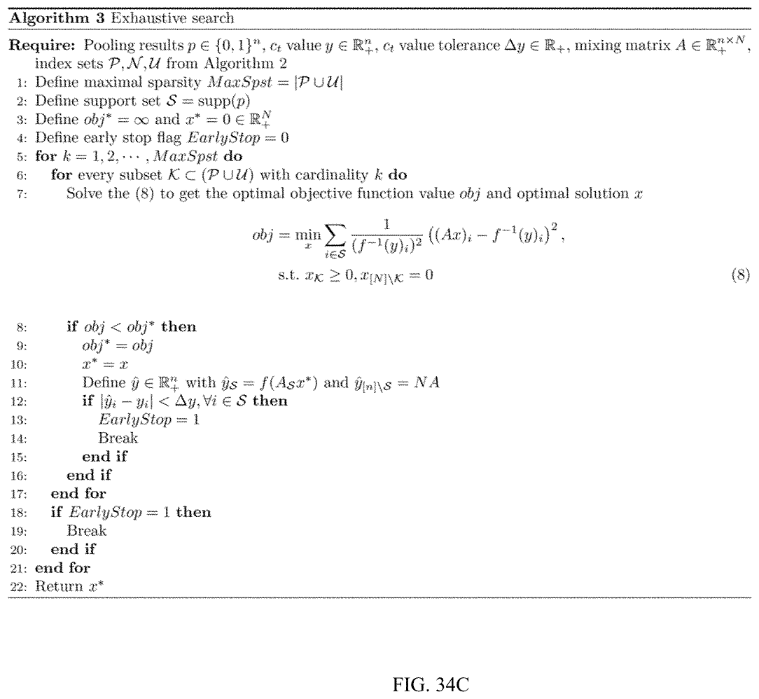

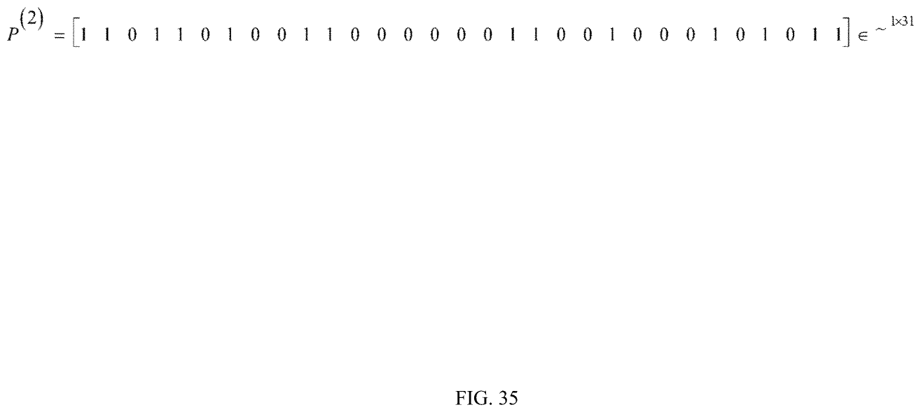

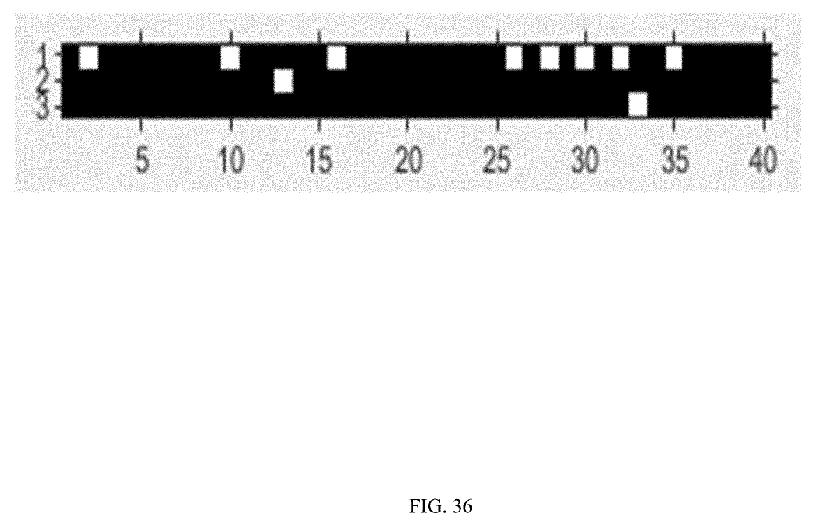

FIGS. 34 to 36, (a), (b), and (c) show the FNR of the pooled testing and the individual testing in log-scale with different probability of outlier error varied from 1% to 15%, and (d), (e), and (f) describe the corresponding FPR. Here, the number of people tested is set to 25, i.e., n=25, and the number of people having Covid-19 virus is varied from 1 to 6 out of 25, i.e., k=1, . . . , 6. The noise level is fixed to 1 e0, From various simulations as shown in FIGS. 34 to 36, the pooled testing lowers the FNR and the FPR as the number of measurements increases. Unlike the pooled testing, the individual testing can reduce the FNR as the number of measurements increases with sacrificing the FPR. This is because of the conservative strategy in the individual testing, which is considering Covid-19 positive if we have at least one positive test result from multiple tests. In some cases where m<n, the individual testing provides lower FPR than that of the pooled testing. This is because the number of tests itself is small in the individual testing, so that there is less chance to have wrong positive results, which leads to the small FPR. Additionally, since we treat the untested case as Covid-19 negative, form m<n, the individual testing has the relatively high FNR in the individual testing. However, for the pooled testing, the FNR and the FPR can be reduced at the same time as the number of measurements increases. This is because as the number of measurements increases, we can recover more accurate results x and e via [symbol] - [symbol] minimization introduced in (7). From these various simulation results with different probability of outlier error, for in <n, we demonstrate that the pooled testing can have lower FNR and FPR than those of the individual testing even in the conservative manner.

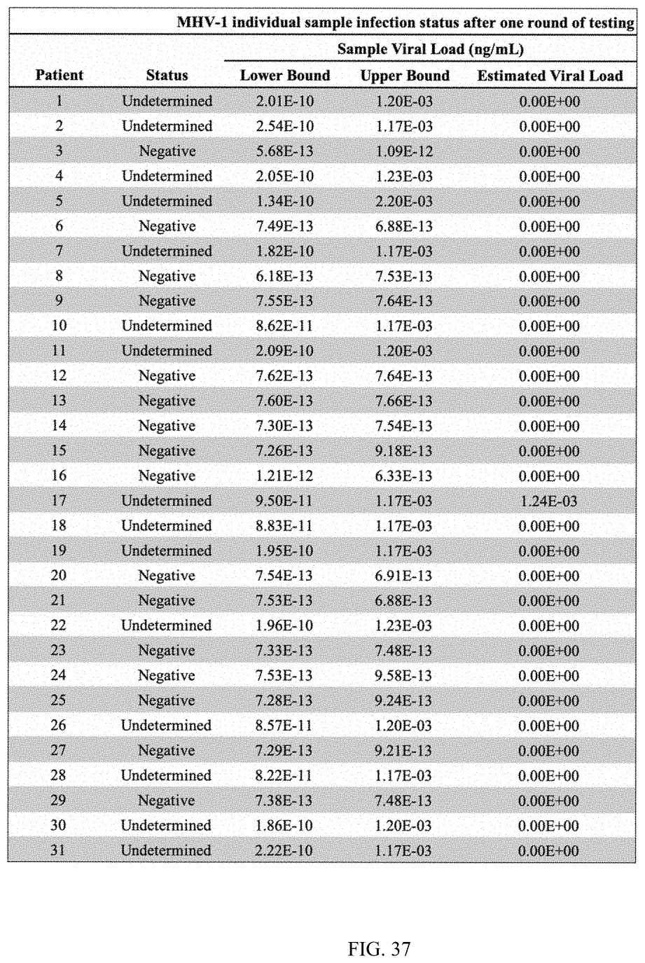

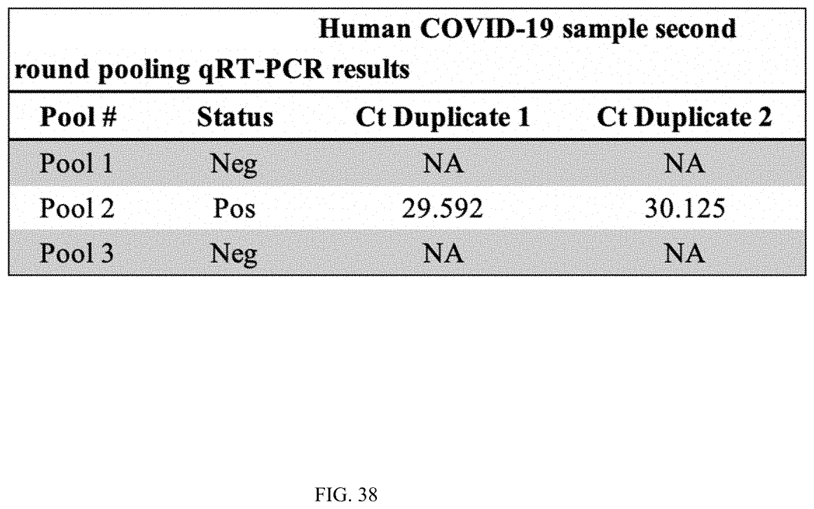

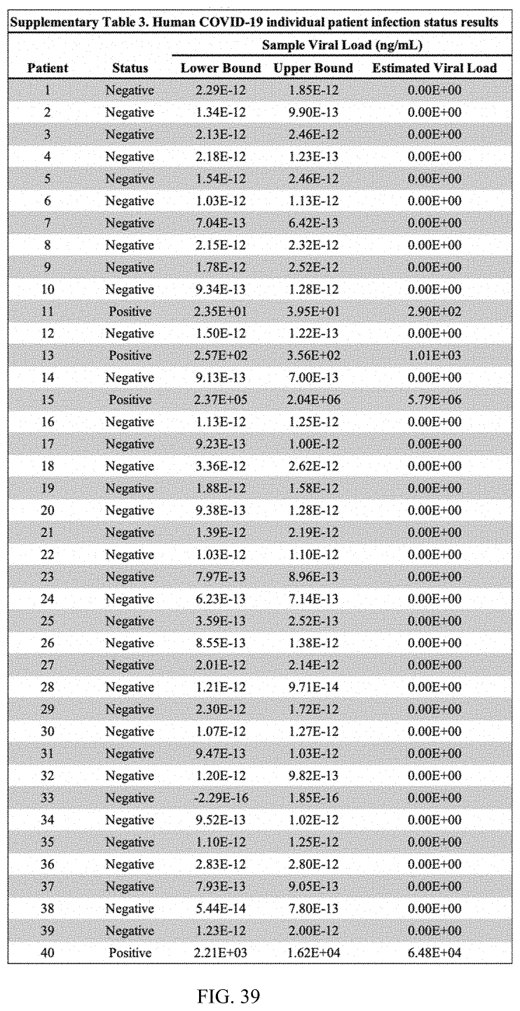

Furthermore, we demonstrate the outperformance of the pooled testing in the Covid-19 testing against the individual testing with more people. FIGS. 37 to 39 show the comparison results in both FNR and FPR as the number of measurements increases between the pooling testing and the individual testing for n=40. In FIGS. 37 to 39, (a), (b), and (c) show the FNR. of the pooled testing and the individual testing with different probability of outlier error from 1% to 15% and different sparsity level from k=1 to k=6. Correspondingly, in FIGS. 34 to 36, (d), (e), and (f) indicate the FPR of the both testing. Through the simulation results shown in FIGS. 37 to 39, with even larger n, it is shown that the pooled testing can identify people having Covid-19 virus more accurately than the individual testing with small number of measurements. Therefore, the pooled testing can have higher throughput than the individual testing. Due to readability, we place most of Figures except for k=3 in the appendix.

### B. Different Noise Levels

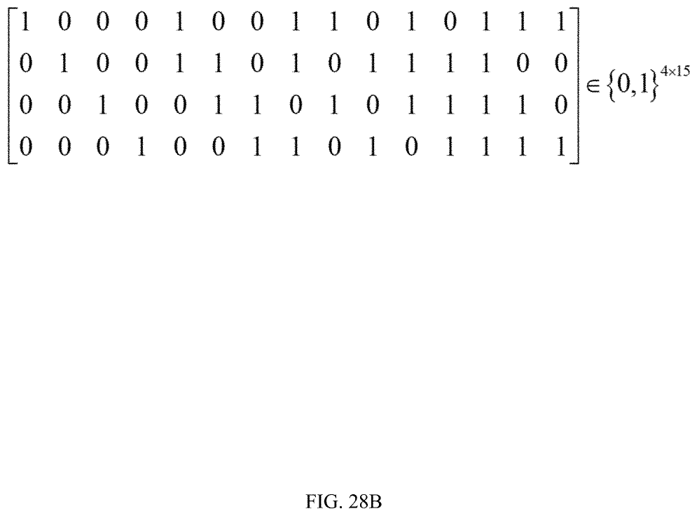

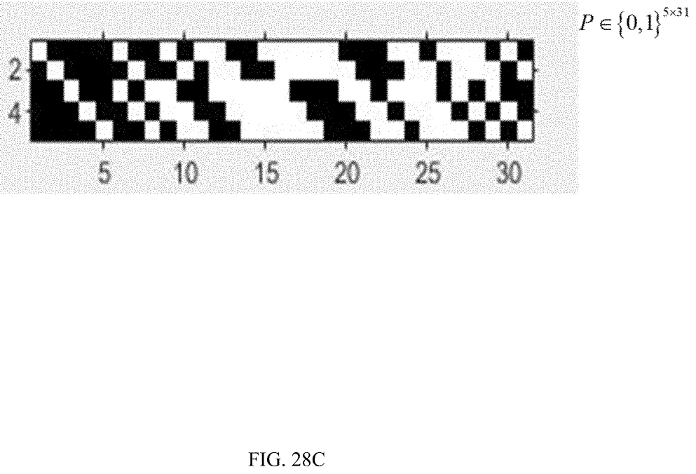

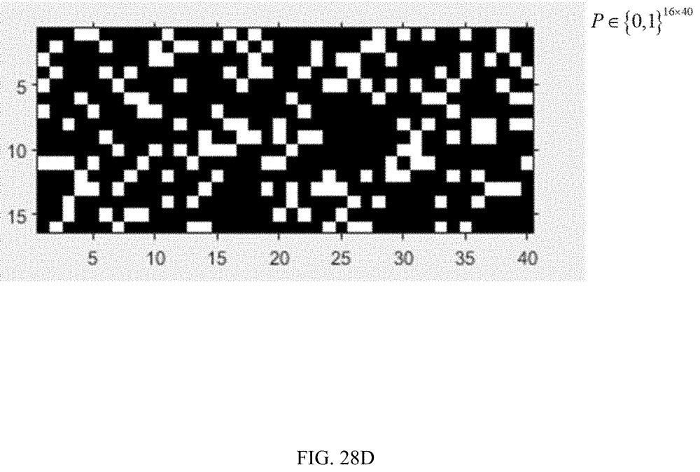

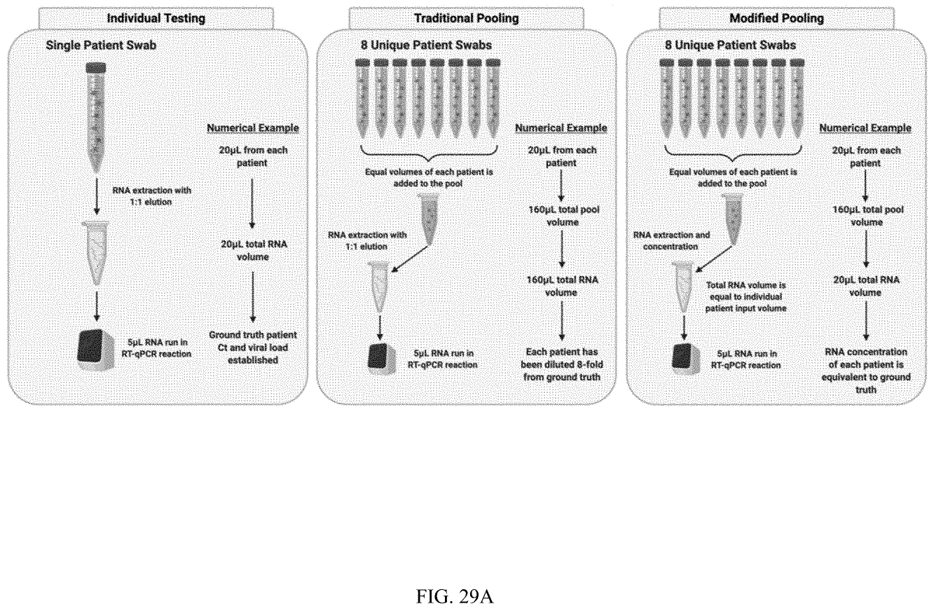

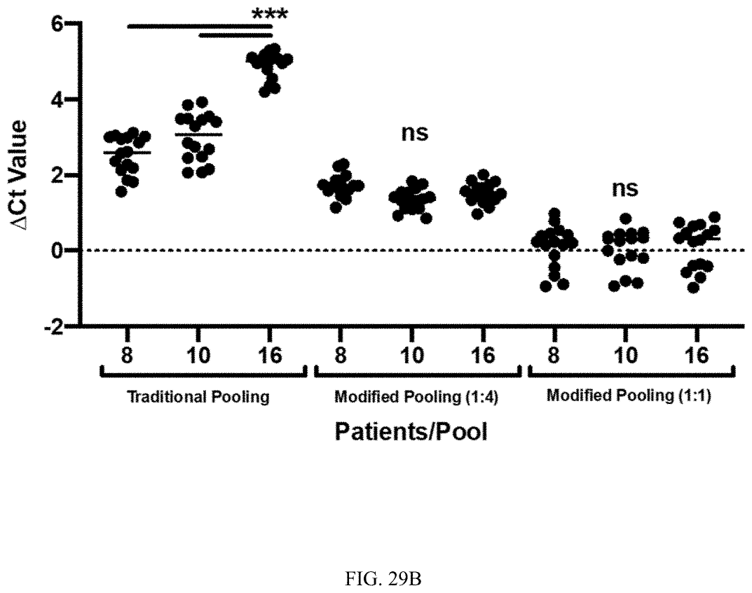

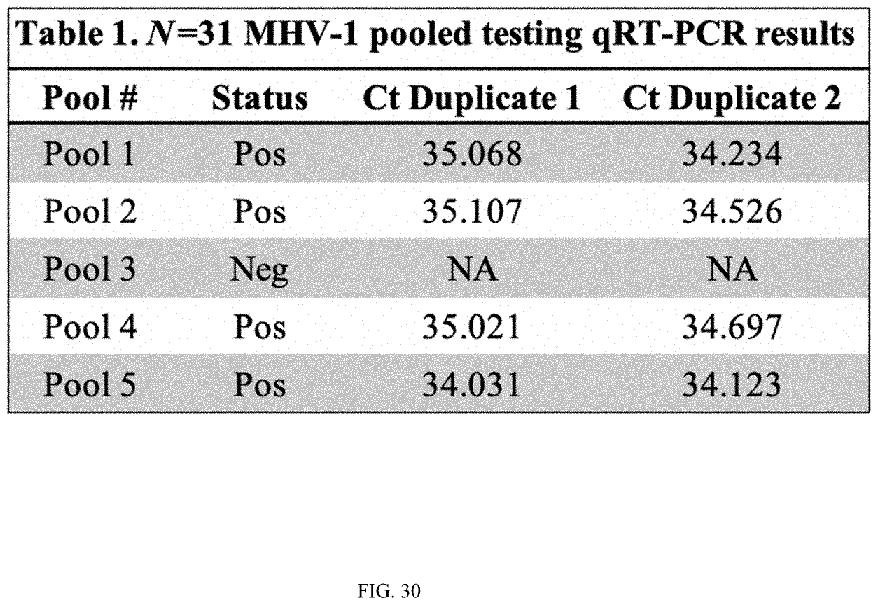

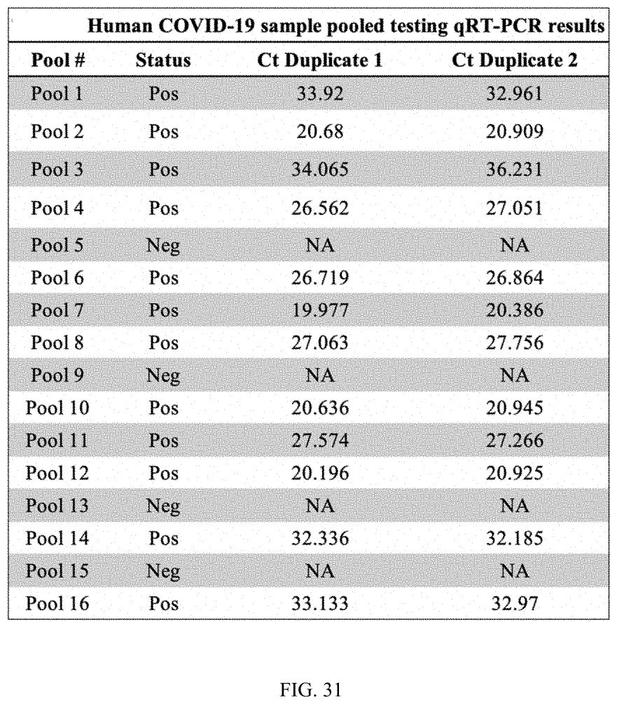

In order to check the Gaussian noise impact, we further run simulations by varying noise level. We vary the Gaussian noise level from 5e-1 to 2e0. We randomly choose 100 trials and record the FNR and the FPR of the pooled testing and the individual testing. Here in the simulations, we set the sparsity level to 3, i.e., k=3, and consider the two probability of outlier error 5% and 15%. FIGS. 28 and 29 illustrate the simulation results in log-scale with Pₒᵤₜ=0.05 when n=25 and n=40 respectively. In addition, FIGS. 30 and 31 show the simulation results in log-scale with Pout=0.15 when n=25 and n=40 respectively. Through the simulation results, it is shown that the individual testing is less suffered from the noise level than the pooled testing. This is because the value of the measurement yᵢ is slightly changed due to the Gaussian noise vᵢ: hence, figuring out the existence of Covid-19 virus in a testee is not much affected. In spite of that, the pooled testing still outperforms the individual testing with various noise level in term of the FNR in every measurement range, and the FPR for m≥n.

### C. Different Sparsity Levels

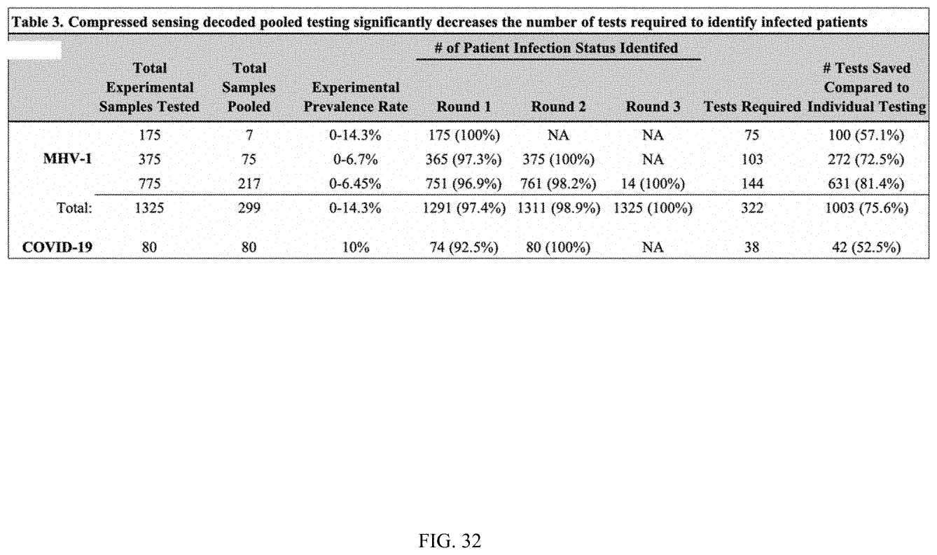

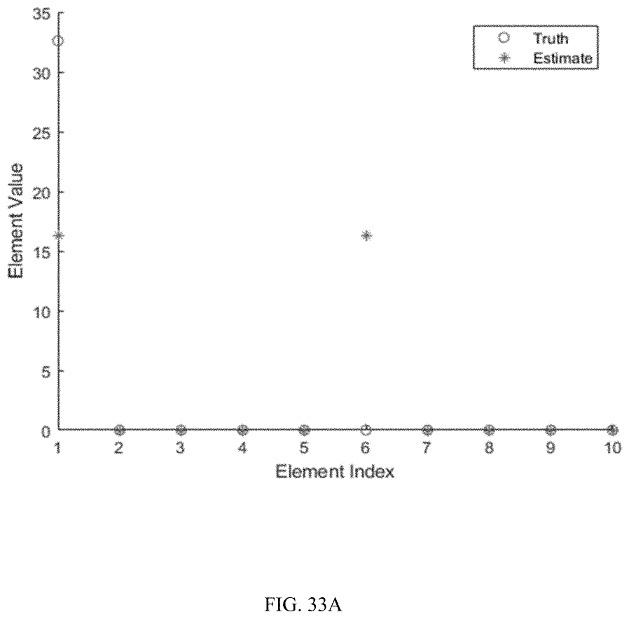

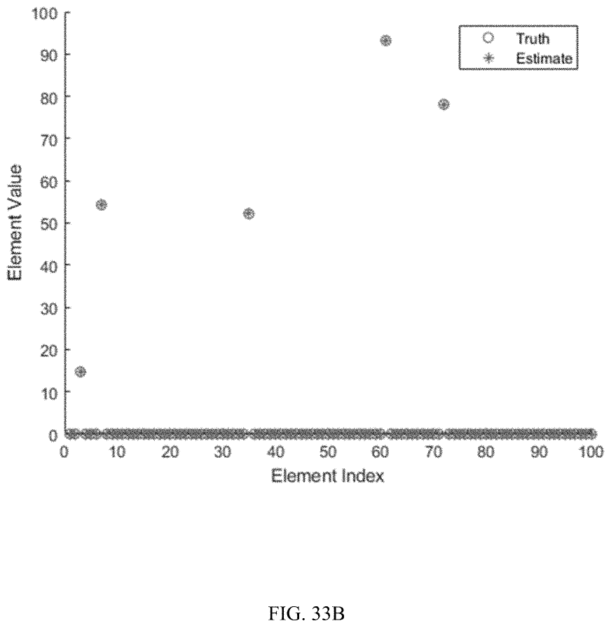

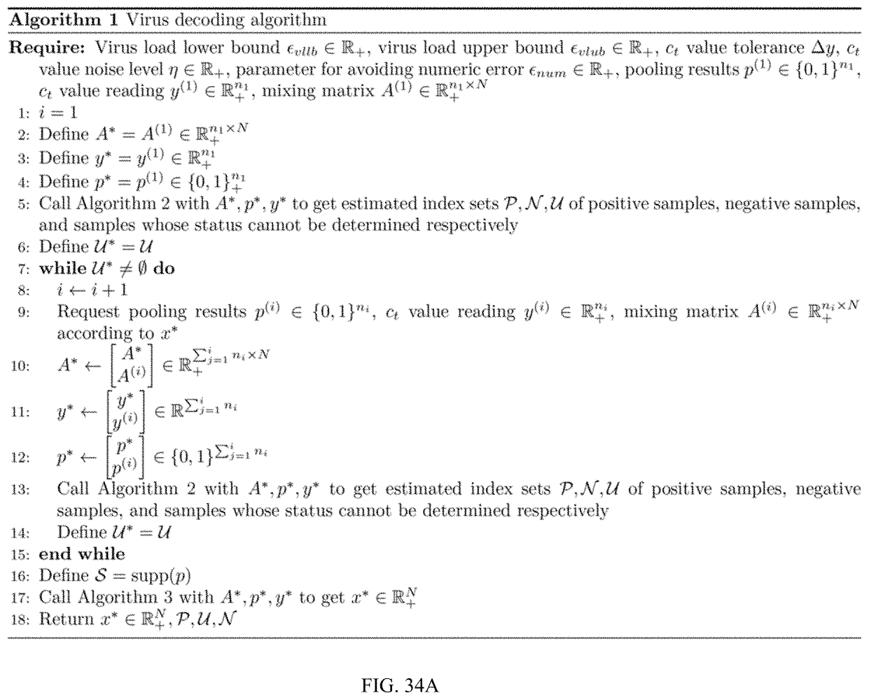

In this subsection, we further run simulations by varying the sparsity level, i.e., the number of people having Covid-19 virus. For these simulations, we set the noise level to 5e−1 and the probability of outlier error Pₒᵤₜ to 0.01. We vary the sparsity level k from 1 to 6. FIGS. 32 and 33 show the FNR and FPR of both the pooled testing and individual testing with different sparsity level when n=25 and n=40 respectively.

### D. Discussion

The overall takeaway from FIGS. 37 to 39 is that the pooled sampling method achieves significantly higher accuracy compared to individual testing. Also in absolute terms, the pooled sampling method is able to provide accurate diagnostic results even when individual. test results are highly noisy. Some specific observations from the simulations are as follows.
In most of the simulations, the pooled sampling method simultaneously achieves lower FPR and FNR than individual sampling. We did not observe even a single instance when the opposite was true i.e. where individual testing outperformed the pooled sampling method in both FPR and FNR.
The FPR for the individual sampling method actually gets worse with increased number of measurements. This is simply an artifact of the individual testing method's conservative strategy in order to prevent miss in Covid-19 positive case. The overall accuracy of the individual testing method does always improve with increased number of measurements when FNR is taken into account along with FPR.
For the pooled sampling method, both FPR and FNR always monotonically decrease with increased number of measurements. (The apparent non-monotonicity in e.g. FIG. 32(f) is simply an artifact of the randomness in the simulations.)

### Part 4: Options, Variations, and Alternatives

Although specific examples have been set forth herein, numerous options, variations, and alternatives are contemplated. For example, although biological testing such as testing for a virus associated with particular antibodies or associated with particular RNA or DNA fragments is described, it is to be understood that the test samples described herein may be of any number of types of materials and the target substance may be practically any substance being tested for.

The methods described herein or aspects thereof may be incorporated into software in the form of instructions stored on a non-transitory computer or machine readable medium which may be used to determine mixing, allocation, and decoding.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments may be described herein as implementing mathematical methodologies including logic or a number of components, modules, or mechanisms. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs).)

The performance of certain of the operations may be distributed among the one or more processors, not only residing within a single machine, but deployed across a number of machines. In some example embodiments, the one or more processors or processor-implemented modules may be located in a single geographic location (e.g., within a hospital, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the disclosure. This description should be read to include one or at least one and the singular also includes the plural unless it is obvious that it is meant otherwise.

The invention is not to be limited to the particular embodiments described herein. In particular, the invention contemplates numerous variations in segmentation. The foregoing description has been presented for purposes of illustration and description. It is not intended to be an exhaustive list or limit any of the invention to the precise forms disclosed. It is contemplated that other alternatives or exemplary aspects are considered included in the invention. The description is merely examples of embodiments, processes, or methods of the invention. It is understood that any other modifications, substitutions, and/or additions can be made, which are within the intended spirit and scope of the invention.

### REFERENCES

[1] E. J. Cand'es and T. Tao, “Decoding by linear programming,” IEEE Transactions on Information Theory, vol. 51, no. 12, pp. 4203-4215, 2005.
[2] D. L. Donoho, “Compressed sensing,” IEEE Transactions on Information Theory, vol. 52, no. 4, pp. 1289-1306, April 2006.
[3] D. Du and F. Hwang, Combinatorial Group Testing and Its Appl, ser. Series on Applied Mathematics Series. World Scientific, 1993. [Online]. Available: http://books.google.com/books?id=b-57lhNsjU8C
[4] W. Xu and B. Hassibi, “Efficient compressive sensing with deterministic guarantees using expander graphs,” in IEEE Information Theory Workshop 2007, 2007, pp. 414-419.
[5] S. Jafarpour, W Xu, B. Hassibi, R. Calderbank. “Efficient and robust compressed sensing using optimized expander graphs”. IEEE Transactions on Information Theory, vol. 55, no. 9, pp. 4299-4308, 2009.
[6] D. Donoho, A. Maleki, A. Montanari. “Message-passing algorithms for compressed sensing”. Proceedings of the National Academy of Sciences, vol. 106, no. 45, pp. 18914-18919, 2009.
[7] M. A. Khajehnejad, A. G. Dimakis, W. Xu, and B. Hassibi, “Sparse recovery of nonnegative signals with minimal expansion,” IEEE Transactions on Signal Processing, vol. 59, no. 1, pp. 196-208, 2011.
[8] E. J. Candes, J. Romberg, and T. Tao, “Robust uncertainty principles: exact signal reconstruction from highly incomplete frequency information,” IEEE Transactions on Information Theory, vol. 52, no. 2, pp. 489-509, February 2006.
[9] D. Donoho, “High-dimensional centrally symmetric polytopes with neighborliness proportional to dimension,” Discrete and Computational Geometry, vol. 35, no. 4, pp. 617-652, 2006.
[10] D. Donoho and J. Tanner, “Thresholds for the recovery of sparse solutions via [symbol] minimization,” in Proceedings of the Conference on Information Sciences and Systems, 2006.
[11] A. Cohen, W. Dahmen, and R. DeVore, “Compressed sensing and best k-term approximation,” J. Amer. Math. Soc. 22 (2009), 211-231, 2008.
[12] W. Xu and B. Hassibi, “Precise stability phase transitions for [symbol] minimization: A unified geometric framework,” IEEE Transactions on Information Theory, vol. 57, no. 10, pp. 6894-6919, October 2011.
[13] A. Juditsky and A. Nemirovski, “On verifiable sufficient conditions for sparse signal recovery via ‘1 minimization,” Math. Programmng, vol. 127, pp. 57-88, 092008.
[14] A. d'Aspremont and L. Ghaoui, “Testing the nullspace property using semidefinite programming,” Mathematical Programming, vol. 127, pp. 123-144, 032011.
[15] M. Cho, K. Mishra, W. Xu. “Computable performance guarantees for compressed sensing matrices”. EURASIP journal on advances in signal processing, 2018(1):16, 2018.
[16] R. Dorfman, “The detection of defective members of large populations,” The Annals of Mathematical Statistics, vol. 14, no. 4, pp. 436-440, 1943.
[17] M. E. Arnold, M. J. Slomka, V. J. Coward, S. Mahmood, P. J. Raleigh, and I. H. Brown, “Evaluation of the pooling of swabs for real-time per detection of low titre shedding of low pathogenicity avian influenza in turkeys,” Epidemiology and Infection, vol. 141, no. 6, pp. 1286-1297, 2013.
[18] S. M. Taylor, J. J. Juliano, P. A. Trottman, J. B. Griffin, S. H. Landis, P. Kitsa, A. K. Tshefu, and S. R. Meshnick, “High-throughput pooling and real-time per-based strategy for malaria detection,” Journal of Clinical Microbiology, vol. 48, no. 2, pp. 512-519, 2010.
[19] A. Schliep, D. C. Torney, and S. Rahmann, “Group testing with dna chips: generating designs and decoding experiments,” in Computational Systems Bioinformatics. CSB2003. Proceedings of the 2003 IEEE Bioinformatics Conference. CSB2003, 2003, pp. 84-91.
[20] H. Q. Ngo and D.-Z. Du, “A survey on combinatorial group testing algorithms with applications to dna library screening,” Discrete mathematical problems with medical applications, vol. 55, pp. 171-182, 2000.
[21] G. K. Atia and V. Saligrama, “Boolean compressed sensing and noisy group testing,” IEEE Transactions on Information Theory, vol. 58, no. 3, pp. 1880-1901, 2012.
[22] J. Haupt, R. Baraniuk, R. Castro, and R. Nowak, “Sequentially designed compressed sensing,” in 2012 IEEE Statistical Signal Processing Workshop (SSP), 2012, pp. 401-404.
[23] E. Candes and T. Tao. “Near-optimal signal recovery from random projections: universal encoding strategies?”, IEEE Transactions on Information Theory, vol. 52, no. 12, pp. 5406-5425, 2006.
[24] A. MacDonald. Scaling up primer and probe kits for Covid-19 testing. wWw.technologynetworks.com, 2020.
[25] E. Emanuel, G. Persad, R. Upshur, B. Thome, M. Parker, A. Glickman, C. Zhang, C. Boyle, M. Smith, and J. Phillips. “Fair allocation of scarce medical resources in the time of Covid-19,” New England Journal of Medicine, 382:2049-2055, 2020.
[26] Gibson, U. E., Heid, C. A., Williams, P. M.: A novel method for real time quantitative rt-per. Genome Research 6(10), 995{1001 (1996). DOI 10.1101/gr.6.10.995.
[27] Hanel, R., Thurner, S.: Boosting test-efficiency by pooled testing strategies for sars-cov-2 (2020)
[28] Hogan, C. A., Sahoo, M. K., Pinsky, B. A.: Sample Pooling as a Strategy to Detect Community Transmission of SARS-CoV-2. JAMA (2020). DOI 10.1001/jama.2020. 5445.
[29] Juditsky, A., Nemirovski, A.: On veriable sufficient conditions for sparse signal recovery via ‘1 minimization. Math. Programmng 127, 57{88 (2008). DOI 10.1007/s10107-010-0417-z
[30] Kralik, P., Ricchi, M.: A basic guide to real time PCR in microbial diagnostics: definitions, parameters, and everything. Frontiers in Microbiology 8, 108 (2017). Publisher: Frontiers
[31] Lamb, L. E., Bartolone, S. N., Ward, E., Chancellor, M. B.: Rapid detection of novel coronavirus (covid-19) by reverse transcription-loop-mediated isothermal amplification. medRxiv (2020). DOI 10.1101/2020.02. 19.20025155.
[32] Lee, V. J., Chiew, C. J., Khong, W. X.: Interrupting transmission of COVID-19: lessons from containment efforts in Singapore. Journal of Travel Medicine (2020). DOI 10.1093/jtm/taaa039.
[33] Nolan, T., Hands, R. E., Bustin, S. A.: Quanti cation of mrna using real-time rt-per. Nature protocols 1(3), 1559 (2006)
[34] Ranney, M. L., Griffeth, V., Jha, A. K.: Critical supply shortages the need for ventilators and personal protective equipment during the covid-19 pandemic. New England Journal of Medicine (2020)
[35] Salathe, M., Althaus, C. L., Neher, R., Stringhini, S., Hodcroft, E., Fellay, J., Zwahlen, M., Senti, G., Battegay, M., Wilder-Smith, A., et al.: Covid-19 epidemic in switzerland: on the importance of testing, contact tracing and isolation. Swiss medical weekly 150(1112) (2020)
[36] Schmid-Burgk, J. L., Li, D., Feldman, D., Slabicki, M., Borrajo, J., Strecker, J., Cleary, B., Regev, A., Zhang, F.: Lamp-seq: Population-scale covid-19 diagnostics using a compressed barcode space. bioRxiv (2020). DOI 10.1101/2020.04.06.025635. URL https://www.biorxiv. org/content/early/2020/04/08/2020.04.06.025635
[37] Scientific, T. F.: Real-time PCR handbook. Nueva York, Estados Unidos de Amrica: Thermo sherScientic (2014)
[38] Shani-Narkiss, H., Gilday, O. D., Yayon, N., Landau, I. D.: Efficient and practical sample pooling high-throughput per diagnosis of covid-19. medRxiv (2020). DOI 10.1101/2020.04.06.20052159. URL https://www.medrxiv.org/content/early/2020/04/07/2020.04.06.20052159
[39] Shental, N., Amir, A., Zuk, O.: Identification of rare alleles and their carriers using compressed se(que)nsing. Nucleic Acids Research 38(19), e179{e179 (2010). DOI 10.1093/nar/gkq675. URL https://doi.org/10.1093/nar/gkq675
[40] Sinnott-Armstrong, N., Klein, D., Hickey, B.: Evaluation of group testing for sars-cov-2 rna. medRxiv (2020). DOI 10.1101/2020.03.27.20043968. URL https://www.medrxiv.org/content/early/2020/03/30/2020.03.27.20043968
[41] Yelin, I., Aharony, N., Shaer-Tamar, E., Argoetti, A., Messer, E., Berenbaum, D., Shafran, E., Kuzli, A., Gandali, N., Hashimshony, T., Mandel-Gutfreund, Y., Halberthal, M., Ge en, Y., Szwarcwort-Cohen, M., Kishony, R.: Evaluation of covid-19 rt-qpcr test in multi-sample pools. medRxiv (2020). DOI 10.1101/2020.03.26.20039438. URL https://www.modrxiv.org/content/early/2020/03/27/2020.03.26.20039438
[42] Zhu, J., Rivera, K., Baron, D.: Noisy pooled per for virus testing (2020)
[43] M. Salath, C. Althaus, R. Neher, S. Stringhini, E. Hodcroft, J. Fellay, M. Zwahlen, G. Senti, M. Battegay, A. Wilder-Smith, I. Eckerle, M. Egger, and N. Low, “COVID-19 epidemic in Switzerland: on the importance of testing, contact tracing and isolation,” Swiss Medical Weekly, vol. 150, p. w20225, 2020.
[44] T. Nolan, R. Hands, and S. Bustin, “Quantification of mRNA using real-time RT-PCR,” Nature Protocols, vol. 1, no. 3, pp. 1559-1582, August 2006.
[45] R. Lequin, “Enzyme immunoassay (EIA)/enzyme-linked immunosorbent assay (ELISA),” Clin Chem, vol. 51, no. 12, pp. 2415-2418, December 2005.
[46] C. Hogan, M. Sahoo, and B. Pinsky, “Sample pooling as a strategy to detect community transmission of SARS-CoV-2,” JAMA, vol. 323, no. 19, pp. 1967-1969, May 2020.
[47] J. Yi, R. Mudumbai, and W. Xu, “Low-cost and high-throughput testing of COVID-19 viruses and antibodies via compressed sensing: system concepts and computational experiments,” arXiv:2004.05759 [cs, eess, math, q-bio], April 2020.
[48] S. Foucart and H. Rauhut, A mathematical introduction to compressive sensing. Birkhuser Basel, 2013, vol. 1, no. 3.
[49] M. Grant, S. Boyd, and Y. Ye, CVX: Matlab software for disciplined convex programming. 2008.

What is claimed is:

1. A method for pooled sample testing for a target substance using compressed sensing, the method comprising:
receiving a plurality of individual samples;
determining a mixing matrix for a plurality of pooled sample mixtures to create by mixing portions of selected ones of the plurality of individual samples;
determining an allocation matrix for the plurality of pooled samples, wherein the allocation matrix allocations portions of each of the plurality of pooled samples for each test;
performing mixing to create the plurality of pooled sample mixtures based on the mixing matrix and the allocation matrix;
performing quantitative tests on the plurality of pooled sample mixtures so as to estimate an amount of the target substance contained within each of the plurality of pooled sample mixtures; and
decoding results of the quantitative tests on the plurality of the pooled sample mixtures using the mixing matrix and the allocation matrix to determine quantitative estimates of amount of the target substance in each of the plurality of individual samples.

2. The method of claim 1 wherein the decoding results of the quantitative tests on the plurality of the pooled sample mixtures further comprises correcting for one or more incorrect test results of the quantitative tests.

3. The method of claim 1 wherein at least a portion of the plurality of pooled sample mixtures are determined after a portion of the quantitative tests are performed to provide for adaptive compressed sensing-based testing.

4. The method of claim 1 wherein the mixing matrix is an expander graph based compressed sensing matrix.

5. The method of claim 1 wherein the results of the quantitative tests are represented in a measurement matrix and wherein the measurement matrix is a sparse bipartite graph based measurement matrix.

6. The method of claim 1 wherein the results of the quantitative tests are represented in a measurement matrix and wherein the measurement matrix is an expander graph based compressed sensing matrix.

7. The method of claim 1 wherein the target substance comprises at least one of a target DNA, a target RNA, and a target protein.

8. The method of claim 1 wherein the target substance is used to infer at least one of virus infections and antibodies.

9. The method of claim 1 wherein the target substance is associated with testing for a COVID-19 virus.

10. The method of claim 1 wherein the performing the quantitative tests comprises performing quantitative PCR (qPCR) tests for virus detection.

11. The method of claim 1 wherein the performing the quantitative tests comprises performing digital PCR (dPCR) tests for virus detection.

12. The method of claim 1 wherein the performing the quantitative tests comprises performing enzyme-linked immunosorbent assay (ELISA) tests for antibody detection.

13. The method of claim 1 wherein the determining the mixing matrix for the plurality of pooled sample mixtures is performing using a computing device.

14. The method of claim 13 wherein the determining an allocation matrix for the plurality of pooled samples is performed using the computing device.

15. The method of claim 11 wherein the decoding the results of the quantitative tests on the plurality of the pooled sample mixtures using the mixing matrix and the allocation matrix to determine the quantitative estimates of amount of the target substance in each of the plurality of individual samples is performed using the computing device.

16. The method of claim 1 wherein the decoding results of the quantitative tests comprises solving a minimization problem based on a [symbol] minimization problem.

17. The method of claim 16 wherein the [symbol] minimization problem is modified to allow that only a small proportion of tests results may be in error.

18. A system pooled sample testing for a target substance using compressed sensing, the system comprising:
a computing device having a memory;
instructions stored on the memory for:
determining a mixing matrix for a plurality of pooled sample mixtures to create by mixing portions of selected ones of a plurality of individual samples;
determining an allocation matrix for the plurality of pooled samples, wherein the allocation matrix allocations portions of each of the plurality of pooled samples for each test; and
decoding results of the quantitative tests on the plurality of the pooled sample mixtures using the mixing matrix and the allocation matrix to determine quantitative estimates of amount of the target substance in each of the plurality of individual samples.

19. A method for pooled sample testing for a target substance using adaptive compressed sensing, the method comprising:
allocating portions of a plurality of individual samples and mixing the portions to provide pooled sample tests;
performing quantitative testing on the pooled sample tests to provide test results;
analyzing the test results and performing additional allocation of portions of the plurality of individual samples and mixing of the portions to provide at least one additional pooled sample test.

20. The method of claim 19 wherein results of the at least one additional pooled sample test provides for certifying correctness of the test results.
